(19) United States
(12) Patent Application Publication
(10) Pub. No.: US 2006/0280798 A1
(43) Pub. Date: Dec. 14, 2006

(54) Nanoparticles for delivery of a pharmacologically active agent

(75) Inventor: Barbara Ensoli, Roma (IT)

Correspondence Address: CLARK & ELBING LLP, 101 FEDERAL STREET, BOSTON, MA, 02110, US

(73) Assignee: ISTITUTO SUPERIORE DI SANITA, ROMA (IT)

(21) Appl. No.: 10/577,973

(22) PCT Filed: Nov. 3, 2004
(86) PCT No.: PCT/EP04/12420

§ 371 Date: Jun. 26, 2006

(30) Foreign Application Priority Data
Nov. 3, 2003 (GB) 0325625.2

(51) Int. Cl. A61K 39/21; A61K 9/14

(52) U.S. Cl. 424/487; 424/184.1; 424/188.1; 977/906; 977/802

(57) ABSTRACT

Core-shell nanoparticles comprising: (a) a core which comprises a water insoluble polymer or copolymer, and (b) a shell which comprises a hydrophilic polymer or copolymer; said nanoparticles being obtainable by emulsion polymerization of a mixture comprising, in an aqueous solution, at least one water-insoluble styrenic, acrylic or methacrylic monomer and specific hydrophylic monomers or copolymers.

### FIELD OF THE INVENTION

The present invention relates to core-shell nanoparticles, processes for preparing them, and their use as carriers able to reversibly bind and deliver pharmacologically active substances, in particular nucleic acids, including natural and modified (deoxy)ribonucleotides (DNA, RNA), oligo(deoxy)nucleotides (ODNs) and proteins, into cells.

### BACKGROUND OF THE INVENTION

DNA vaccines are known to induce immune responses and protective immunity in many animal models of infectious diseases. In human clinical trials, certain DNA vaccines have been shown to induce immune responses, but multiple immunizations of high doses of DNA were required. Therefore, in order to provide protective efficacy in humans, the potency of DNA vaccines needs to be increased.

During the past decade, new therapeutic approaches introducing genetic materials (such as genes, antisense oligonucleotides and triple-helix-forming oligonucleotides) into intact cells have shown rapid progress both fundamentally and clinically in gene therapy. Many types of synthetic carriers, including liposomes, polymers and polymeric particles have been studied to deliver exogenous genetic materials into cells in a cellular specific or non specific manner. Recently biodegradable or biocompatible polymeric nano-microparticles have been studied as a potential carrier for genetic materials. Advantages of biocompatible polymeric particles as gene delivery carriers include: 1) they are relatively inert and biocompatible; 2) their biological behaviour can be regulated by controlling the size and surface properties; and 3) preparation, storage, and handling are relatively easy. The size and shape of the resulting formulation can also remain homogeneous and uniform, compared to the formulations based on liposomes or polycations.

Controlled delivery systems consisting of biocompatible polymers can potentially protect DNA or proteins from degradation until they are both released and delivered to the desired location at predetermined rates and durations to generate an optimal immune response. The combination of slow release and depot effect may reduce the amount of antigens used in the vaccine and eliminate the booster shots that are necessary for the success of many vaccinations. Moreover, a controlled delivery system can efficiently direct antigens into antigen-presenting cells (APCs) to generate both cellular and humoral responses.

Bertling et al. (Biotechnol. Appl. Biochem. (1991) 13, 390-405) prepared nanoparticles from polycyanoacrylate in the presence of DEAE-dextran. These nanoparticles exhibited a strong DNA binding capacity and resistance against DNAse I degradation, although the biological activity of the plasmid DNA was not observed presumably due to the strong binding of the DNA to particles. Poly(alkyl cyanoacrylate) nanoparticles were also evaluated as an oligonucleotide carrier, and their physical stability and biological efficacy of antisense oligonucleotides were found to be greatly enhanced in this formulation (Cortesi et al., Int. J. Pharm. (1994), 105, 181-186; Chavany et al., Pharm. Res. (1994), 11, 1370-1378). Poly(isohexyl cyanoacrylate) nanoparticles were recently used to adsorb cholesterol-oligonucleotide conjugates on their surface via hydrophobic interaction, where a sequence specific antisense effect was observed only when the oligonucleotide was associated with the nanoparticles (Godard et al., Eur. J. Biochem. (1995), 232, 404-410). In the studies mentioned above, the majority of the surface of the particles was probably occupied by poly(oligo)nucleotides and it was difficult to modify the particle surfaces with functional molecules, such as ligand moieties, to modulate biodistribution.

Poly(lactide-co-glycolide) (PLG) microparticles have been intensively studied for vaccine delivery, since the polymer is biodegradable and biocompatible and has been used to develop several drug delivery systems. In addition, PLG microparticles have also been used for a number of years as delivery systems for entrapped vaccine antigens. More recently, PLG microparticles have been described as a delivery system for entrapped DNA vaccines. Nevertheless, recent observations have shown that DNA is damaged during microencapsulation, leading to a significant reduction in supercoiled DNA. Moreover, the encapsulation efficiency is often low. O'Hagan et al. (Proc. Natl. Acad. Sci. U.S.A. (2000), 97(2), 811-816; Journal of Virology (2001), 75, 9037-9043) first developed a novel approach of adsorbing DNA onto the surface of PLG microparticles to avoid the problems associated with microencapsulation of DNA. Due to the lipophilic nature of the polymer, the addition of hydrophobic cations to the suspension is required to allow DNA binding on the microparticle surface. This approach was shown to increase the potency of DNA vaccines in several animal species, such as guinea pigs and rhesus macaques. However, the hydrophobic cation is not covalently bound to the microparticle surface. Moreover, it exhibits toxicity on cell cultures at the high concentration required. A way to produce charged polymeric microparticles able to adsorb directly protein onto their surface was developed also by O'Hagan et al. (J. Control. Release (2000), 67, 347-356). They prepared anionic microparticles through the inclusion of an anionic detergent, sodium dodecyl sulphate (SDS), in the microparticle preparation process. The anionic microparticles were capable of adsorbing recombinant p-55 gag protein from HIV. Again, the anionic material is not covalently bound to the surface of the microparticle.

Duracher et al., Langmuir (2000) 16, 9002-9008 describe the adsorption of modified HIV-1 capsid p24 protein onto thermosensitive and cationic core-shell poly(styrene)-poly (N-isopropylacrylamide) particles. A two-stop procedure was used to make the particles; in the first step batch polymerisation of styrene and N-isopropylacrylamide (NIPAM) was carried out, and the second step, combining emulsifier-free emulsion and precipitation polymerization, consisted of adding a mixture of NIPAM, amino ethylmethacrylate hydrochloride and, as a cross-linker, methylene bisacrylamide. The shell is cross-linked and in the form of a hydrogel.

### SUMMARY OF THE INVENTION

One of the aims of the present invention is to develop biocompatible polymeric carriers able to reversibly bind and deliver pharmacologically active substances, such as nucleic acids intact into cells. Another aim of the invention is to develop stealth carriers, able to avoid recognition by the phagocytic cells, and to last longer in the bloodstream.

The present invention accordingly provides core-shell nanoparticles comprising:

(a) a core which comprises a water insoluble polymer or copolymer, and

(b) a shell which comprises a hydrophilic polymer or copolymer;

said nanoparticles being obtainable by emulsion polymerization of a mixture comprising, in an aqueous solution, at least one water-insoluble monomer and:

(i) a monomer of formula (I): 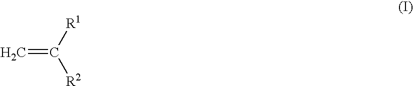
wherein
R¹ represents hydrogen or methyl, and
R² represents, —COOAOH, —COO—A—NR⁹R¹⁰ or —COO—A—N⁺R⁹R¹⁰R¹¹X⁻, in which A represents C₁₋₂₀ alkylene, R⁹, R¹⁰ and R¹¹ each independently represent hydrogen or C₁₋₂₀ alkyl and X represents halogen, sulphate, sulphonate or perchlorate, and
a water-soluble polymer of formula (II) 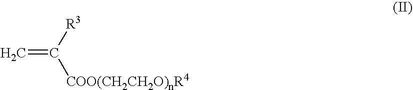
wherein
R³ represents hydrogen or methyl,
R⁴ represents hydrogen or C₁₋₂₀ alkyl, and
n is an integer such that the polymer of formula (I) has a number-average molecular weight of at least 1000; or

(ii) a hydrophilic copolymer which comprises repeating units of formulae (III) and 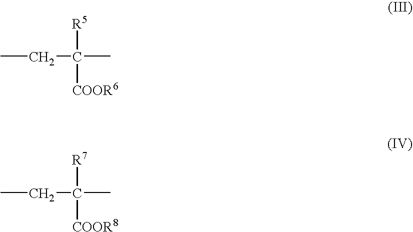
(IV):
wherein
R⁵ and R⁷ each independently represent hydrogen or methyl,
R⁶represents hydrogen, —A—NR⁹R¹⁰ or —A—N⁺R⁹R¹⁰R¹¹X⁻, in which A represents C₁₋₂₀ alkylene,
R⁹, R¹⁰ and R¹¹ each independently represent hydrogen or C₁₋₂₀ alkyl and X represents halogen, sulphate, sulphonate or perchlorate and
R⁸represents C₁₋₁₀ alkyl.

The invention further provides:

a process for preparing the nanoparticles of the invention;

nanoparticles of the invention which further comprise a pharmacologically active agent, such as a pharmaceutical for therapy or diagnosis, adsorbed at the surface of the nanoparticles (hereinafter described as “pharmacologically active nanoparticles”). Preferably the pharmacologically active agent is an antigen, more preferably a disease-associated antigen. Such nanoparticles are hereinafter described as “antigen-containing nanoparticles”;

a process for preparing the pharmacologically active nanoparticles particularly the antigen-containing nanoparticles of the invention;

a pharmaceutical composition comprising the pharmacologically active nanoparticles of the invention;

a method of generating an immune response in an individual, said method comprising administering the antigen-containing nanoparticles of the invention in a therapeutically effective amount;

a method of preventing or treating HIV infection or AIDS, said method comprising administering the pharmacologically active nanoparticles particularly the antigen-containing nanoparticles of the invention in a therapeutically effective amount;

pharmacologically active nanoparticles particularly the antigen-containing nanoparticles of the invention for use in a method of treatment of the human or animal body by therapy or a diagnostic method practised on the human or animal body;

use of the antigen-containing nanoparticles of the invention for the manufacture of a medicament for generating an immune response in an individual; and

use of the pharmacologically active nanoparticles particularly the antigen-containing nanoparticles of the invention for the manufacture of a medicament for preventing or treating HIV infection or AIDS.

### BRIEF DESCRIPTION OF THE DRAWINGS

FIG. 1 is a schematic illustration of the structure of a core-shell nanoparticle obtainable by emulsion polymerization of a water insoluble monomer in an aqueous solution comprising a monomer of formula (I) and a polymer of formula (II).

FIG. 2 is a scanning electron micrograph of nanoparticle sample PEG32 obtained in Example 1.

FIG. 3 is an SEM micrograph of sample ZP2 obtained in Example 2.

FIG. 4 shows nanoparticle size as a function of concentration of non ionic polymer 2 in Example 2.

FIG. 5 shows the quaternary ammonium group amount per gram of nanoparticles in the sample series of Example 2 as a function of concentration of non ionic comonomer.

FIG. 6 is a scanning electron micrograph of nanoparticle sample M1 obtained in Example 3.

FIG. 7 is an SEM micrograph of sample MA7 obtained in Example 5.

FIGS. 8A and 8B are a linear (FIG. 8A) and a logarithmic plot (FIG. 8B) of nanoparticle size as a function of the MMA concentration for the nanoparticles of Example 5.

FIG. 9 shows the carboxylic group amount on the nanoparticle sample series MAₙ of Example 5 as a function of the nanoparticle diameter.

FIG. 10 illustrates ODN adsorption on the nanoparticles obtained in Example 6 as a function of ODN concentration.

FIG. 11 shows ODN adsorption on pegylated nanoparticles ZP3 and PEG32.

FIG. 12 shows DNA adsorption on PEG 32 and ZP3.

FIG. 13 shows the stability of the DNA/PEG32 complex in PBS buffer.

FIG. 14 shows the time dependent release of DNA from PEG 32 nanoparticle in the presence of 1M NaCl phosphate buffer (pH 7.4).

FIG. 15 shows how the adsorption of trypsin varies on MA7 nanoparticles.

FIG. 16 shows how PCS and ζ-potential vary with binding of a model protein (trypsin) to MA7 acid nanoparticles.

FIG. 17 shows DNA/nanoparticles adsorption and release kinetics. For adsorption kinetics, nanoparticles PEG3 and PEG32, resuspended at 10 mg/ml in 20 mM sodium phosphate buffer (pH 7.4), were incubated with increasing amounts of pCV-0 plasmid DNA (10-250 μg/ml), stirred for 2 hours at room temperature and centrifuged. The supernatants were collected, filtered and UV absorbance was measured at 260 nm to determine the amount of unbound DNA. The results are represented as (A) adsorption efficiency (%), calculated as 100×[(administered DNA)−(unbound DNA)/(administered DNA)], and (B) as DNA (μg) loading per mg of nanoparticles. For release kinetics, DNA/nanoparticle complexes were prepared in 20 mM sodium phosphate buffer (pH 7.4) at the ratio of 25 μg of DNA/mg of nanoparticles/ml (DNA/PEG3), and with 10 and 100 μg of DNA/10 mg of nanoparticles/ml (DNA/PEG32). After incubation, complexes were collected by centrifugation, resuspended in 1 M NaCl/20 mM sodium phosphate buffer, and incubated at 37° C. At different time intervals, samples were centrifuged and supernatants analysed by agarose gel electrophoresis to determine the amount of DNA released from PEG3 (C, D) and PEG32 (E, F) complexes. The results represent the percentage (%) of DNA released from the complexes, determined as 100×(released DNA/bound DNA) (C, E). One representative gel of DNA released from each DNA/nanoparticle complex, prepared with 1 μg of DNA, is shown (D, F). Plasmid pCV-0 (0.1 and 0.5 μg) was run as the control in each gel.

FIG. 18 shows the evaluation of cell proliferation in the presence of the PEG3 and PEG32 nanoparticles. HL3T1 cells were cultured for 96 hours with increasing amounts of PEG3 (20-400 μg/ml) and PEG32 (50-500 μg/ml), and cell proliferation measured using a colorimetric MTT-based assay. Controls were represented by untreated cells (None). Results are expressed as the mean (±SD) of sextuples.

FIG. 19 shows the analysis of cellular uptake. HL3T1 cells were cultured in the presence of PEG3-fluo nanoparticles (40 μg) alone (A and B) or associated with 1 μg of pCV-tat DNA (C and D). After 2 (A and C) and 24 (B and D) hours incubation, cells were fixed with paraformaldheyde and observed at a confocal laser scanning microscope.

FIG. 20 shows the analysis at the site of injection of cellular uptake of PEG3-fluo nanoparticles, 15 (panel A) and 30 (panel B) minutes after inoculation. For the same microscopic field, green (nanoparticle) and blue (nuclei) images were taken and overlapped as described in matherials and methods. Magnification 100×.

FIG. 21 shows that polymeric nanoparticles deliver and release functional DNA intracellularly. (A) HeLa cells were incubated with 1 or 10 μg of pGL2-CMV-Luc basic DNA alone or adsorbed onto PEG3 (ratio 25 μg/mg/ml) and PEG32 (ratio 10 or 100 μg/10 mg/ml) nanoparticles. Complexes were prepared as described in matherials and methods and immediately added to the cells. (B) Cells were incubated with 10 μg of pGL2-CMV-Luc basic DNA alone or adsorbed onto PEG32 (ratio 100 μg/10 mg/ml) nanoparticles prepared, as described in matherials and methods, and immediately added to the cells (DNA/PEG32 fresh) or lyophilized, stored at room temperature for 1 month, resuspended for 1 hour at room temperature and added to the cells (DNA/PEG32 lyophilized). In A and B, after 48 hours, luciferase gene expression was measured on cell extracts normalized to total protein content, as described in matherials and methods. Results are the mean of two independent experiments, and are expressed as luciferase light units.

FIG. 22 shows the analysis of CTL response to Tat. B-depleted splenocytes were co-cultivated with Balb/c 3T3-Tat expressing cells for five days, and tested for cytolytic activity against P815 target cells pulsed with Tat peptides containing computer predicted CTL epitopes The percentage (%) of specific lysis is reported.

FIG. 23 shows the histologic findings after injection of DNA/PEG32 complexes by the i.m. route. An inflammatory reaction was observed with variable intensity in the endomysial connective tissue (panels A, B, C) with a mild macrophage cell infiltration without degenerative alterations of muscle fibers (panel B), or sometimes with a more intense mononuclear cell infiltration which caused regressive changes (panel C). Sometimes the macrophages were also found in the adipose tissue surrounding the injection site (panel D). Hematoxylin—Eosin staining. Magnification: 5× (panel A); 20× (panels B and C); 10× (panel D).

FIG. 24 shows the evaluation of cell proliferation in the presence of ZP3 nanoparticles. HL3T1 cells were cultured for 96 hours with increasing amounts of ZP3 (500-10.000 μg/ml) and cell proliferation measured using a colorimetric MTT-based assay. Controls were represented by untreated cells (None). Results are expressed as the mean (±SD) of sextuples.

FIG. 25 shows the analysis of in vitro cytotoxicity of MA7 nanoparticles. HL3T1 cells were cultured for 96 hours in the presence of increasing amounts of MA7 alone (10-500 μg/ml) (left panel) or with the same doses of MA7 bound to Tat protein (1 μg/ml) (right panel). Controls were represented by untreated cells (none) or cells cultured with Tat alone (1 μg/ml) (Tat). Results are the mean of sextupled wells (±SD).

FIG. 26 shows the analysis of the biological activity of Tat complexed with MA7 nanoparticles. HL3T1 cells, containing an integrated copy of plasmid HIV-1-LTR-CAT, where expression of the chloramphenicol acetyl transferase (CAT) reporter gene is driven by the HIV-1 LTR promoter and occurs only in the presence of biologically active Tat protein, were incubated with increasing amounts of Tat (0.125, 0.5 and 1 μg/ml) bound to MA7 nanoparticles (30 μg/ml) (upper panel) or with the same doses of Tat alone (lower panel) in presence of 100 μM chloroquine. Controls were represented by untreated cells (none). After 48 hours, CAT activity was measured on cell extracts normalized to the same protein content. Results are the mean (±SD) of three independent experiments.

### BRIEF DESCRIPTION OF SEQUENCE LISTING

SEQ ID NO: 1 shows the nucleotide sequence that encodes the full length. HIV-1 Tat protein from HTLV-III, BH10 CLONE, CLADE B.

SEQ ID NO: 2 shows the 102 amino acid sequence of full length HIV-1 Tat protein from HILV, BH10 CLONE CLADE B.

SEQ ID NOs: 3 to 32 show the nucleotide and amino acid sequences of variants of the full length HIV-1 Tat protein isolated from HTLV-III, BH10 CLONE, CLADE B. The length and sequence of Tat varies depending on the viral isolate.

SEQ ID NO: 3 shows the nucleotide sequence that encodes the shorter version of HIV-1 Tat protein (BH10).

SEQ ID NO: 4 shows the 86 amino acid shorter version of HIV-1 Tat protein (BH10). This sequence corresponds to residues 1 to 86 of SEQ ID NO: 1.

SEQ ID NO: 5 shows the nucleotide sequence that encodes the cysteine 22 mutant of BH10 (SEQ ID NO: 4).

SEQ ID NO: 6 shows the 86 amino acid cysteine 22 mutant of BH10 (SEQ ID NO: 4).

SEQ ID NO: 7 shows the nucleotide sequence that encodes the lysine 41 mutant of BH10 (SEQ ID NO: 4).

SEQ ID NO: 8 shows the 86 amino acid lysine 41 mutant of BH10 (SEQ ID NO: 4).

SEQ ID NO: 9 shows the nucleotide sequence that encodes the RGDΔ mutant of BH10 (SEQ ID NO: 4).

SEQ ID NO: 10 shows the 83 amino acid RGDΔ mutant of BH10 (SEQ ID NO: 4).

SEQ ID NO: 11 shows the nucleotide sequence that encodes the lysine 41 RGDΔ mutant of BH10 (SEQ ID NO: 4).

SEQ ID NO: 12 shows the 83 amino acid lysine 41 RGDΔ mutant of BH10 (SEQ ID NO: 4).

SEQ ID NO: 13 shows the nucleotide sequence that encodes the consensus_A-A1-A2 variant of HIV-1 Tat protein.

SEQ ID NO: 14 shows the 101 amino acid consensus_A-A1-A2 variant of HIV-1 Tat protein.

SEQ ID NO: 15 shows the nucleotide sequence that encodes the consensus_B variant of HIV-1 Tat protein.

SEQ ID NO: 16 shows the 101 amino acid consensus_B variant of HIV-1 Tat protein.

SEQ ID NO: 17 shows the nucleotide sequence that encodes the consensus_C variant of HIV-1 Tat protein.

SEQ ID NO: 18 shows the 101 amino acid consensus_C variant of HIV-1 Tat protein.

SEQ ID NO: 19 shows the nucleotide sequence that encodes the consensus_D variant D of HIV-1 Tat protein.

SEQ ID NO: 20 shows the 86 amino acid consensus_D variant of the HIV-1 Tat protein.

SEQ ID NO: 21 shows the nucleotide sequence that encodes the consensus_F1-F2 variant of HIV-1 Tat protein.

SEQ ID NO: 22 shows the 101 amino acid consensus_F1-F2 variant of HIV-1 Tat protein.

SEQ ID NO: 23 shows the nucleotide sequence that encodes the consensus_G variant of the HIV-1 Tat protein.

SEQ ID NO: 24 shows the 101 amino acid consensus_G variant of the HIV-1 Tat protein.

SEQ ID NO: 25 shows the nucleotide sequence that encodes the consensus_H variant of the HIV-1 Tat protein.

SEQ ID NO: 26 shows the 86 amino acid consensus_H variant of the HIV-1 Tat protein.

SEQ ID NO: 27 shows the nucleotide sequence that encodes the consensus_CRF01 variant of the HIV-1 Tat protein.

SEQ ID NO: 28 shows the 101 amino acid consensus_CRF01 variant of the HIV-1 Tat protein.

SEQ ID NO: 29 shows the nucleotide sequence that encodes the consensus_CRF02 variant of the HIV-1 Tat protein.

SEQ ID NO: 30 shows the 101 amino acid consensus_CRF02 of the HIV-1 Tat protein.

SEQ ID NO: 31 shows the nucleotide sequence that encodes the consensus_O variant of HIV-1 Tat protein.

SEQ ID NO: 32 shows the 115 amino acid consensus_O variant of the HIV-1 Tat protein.

SEQ ID NO: 33 shows one of the synthetic peptides used for anti-Tat IgG epitope mapping. This sequence corresponds to residues 1-20 of SEQ ID NOs: 2 and 4.

SEQ ID NO: 34 shows one of the synthetic peptides used for anti-Tat IgG epitope mapping. This sequence corresponds to residues 21-40 of SEQ ID NOs: 2 and 4.

SEQ ID NO: 35 shows one of the synthetic peptides used for anti-Tat IgG epitope mapping. This sequence corresponds to residues 36-50 of SEQ ID NOs: 2 and 4.

SEQ ID NO: 36 shows one of the synthetic peptides used for anti-Tat IgG epitope mapping. This sequence corresponds to residues 46-60 of SEQ ID NOs: 2 and 4.

SEQ ID NO: 37 shows one of the synthetic peptides used for anti-Tat IgG epitope mapping. This sequence corresponds to residues 56-70 of SEQ ID NOs: 2 and 4.

SEQ ID NO: 38 shows one of the synthetic peptides used for anti-Tat IgG epitope mapping. This sequence corresponds to residues 52-72 of SEQ ID NOs: 2 and 4.

SEQ ID NO: 39 shows one of the synthetic peptides used for anti-Tat IgG epitope mapping. This sequence corresponds to residues 65-80 of SEQ ID NOs: 2 and 4.

SEQ ID NO: 40 shows one of the synthetic peptides used for anti-Tat IgG epitope mapping. This sequence corresponds to residues 73-86 of SEQ ID NOs: 2 and 4.

### DETAILED DESCRIPTION OF THE INVENTION

It is to be understood that this invention is not limited to particular pharmacologically active agents or antigens. It is also to be understood that different applications of the disclosed methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “an antigen” includes a mixture of two or more such antigens, reference to “a nanoparticle” includes reference to mixtures of two or more nanoparticles and vice versa, reference to “a target cell” includes two or more such cells, and the like.

All publications, patents and patent applications cited herein, whether supra or infra, are hereby incorporated by reference in their entirety.

The invention provides nanoparticles which may be used for delivering a pharmacologically-active agent, particularly an antigen to target cells. The nanoparticles may have pharmacologically active agent adsorbed or fixed onto their external surface.

The nanoparticles of the invention have a core-shell structure, in which the inner core contains a water-insoluble polymer or copolymer. and the outer shell contains a hydrophilic polymer or copolymer. The shell contains functional groups which are charged or ionic or ionisable. Preferably they are ionic or ionisable at physiological pH, for example at a pH in the range from 7.2 to 7.6 and preferably at about 7.4. The nanoparticles are obtainable by emulsion polymerization of a water-insoluble monomer in an aqueous solution comprising a monomer of formula (I) and a polymer of formula (II), or comprising a hydrophilic copolymer which comprises repeating units of formulae (III) and (IV). The water-insoluble monomer is polymerized to form the core. The shell is formed by the monomer of formula (I) and polymer of formula (II), or by the hydrophilic copolymer which comprises repeating units of formulae (III) and (IV). The external nanoparticle surface is typically a hydrophilic shell that comprises ionic, or ionisable chemical groups. The nanoparticle surface may have an overall positive or negative charge. The nanoparticles preferably have a net positive or negative charge over their entire external surface. The surface charge density typically varies across the surface of the nanoparticles.

The shell and core of the nanoparticles may be composed of a biocompatible and biodegradable polymeric material. The term “biocompatible polymeric material” is defined as a polymeric material which is not toxic to an animal and not carcinogenic. The material is preferably biodegradable in the sense that it should degrade by bodily processes in vivo to products readily disposable by the body and should not accumulate in the body. On the other hand, where the nanoparticles are being inserted into a tissue which is naturally shed by the organism (e.g. sloughing of the skin), the material need not be biodegradable.

The water-insoluble polymer or copolymer used in the core of the nanoparticles of the invention may be any water-insoluble polymer or copolymer obtainable by emulsion polymerization of at least one water-insoluble styrenic, acrylic or methacrylic monomer. Suitable materials include, but are not limited to, polyacrylates, polymethacrylates and polystyrenes and acrylic or methacrylic or styrenic copolymers. When the core comprises a water-insoluble copolymer, the emulsion polymerisation process may use more than one comonomer.

Thus the water-insoluble polymer or copolymer in the core is preferably formed from the polymerization of at least one monomer of formula V: 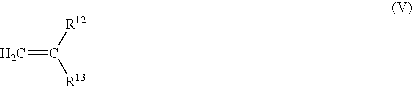
wherein R¹² represents hydrogen or methyl

and R¹³ represents phenyl, —COOR¹⁴, —COCN or CN

in which R¹⁴ is hydrogen or C₁₋₂₀ alkyl

The term “poly(meth)acrylate” as used herein encompasses both polyacrylates and polymethacrylates. Likewise the term “(meth)acrylate” encompasses both acrylates and methacrylates.

Preferred poly(meth)acrylates which may be used as core materials include poly(alkyl (meth)acrylates), in particular poly(C₁₋₁₀ alkyl (meth)acrylates), and preferably poly(C₁₋₆ alkyl (meth)acrylates) such as poly(methyl acrylate), poly(methyl methacrylate), poly(ethyl acrylate), and poly(ethyl methacrylate). Poly(methyl methacrylate) (PMMA) is especially preferred as the core material. PMMA has been used in surgery for over 50 years and is slowly biodegradable (about 30% to 40% of the polymer per year) in the form of nanoparticles.

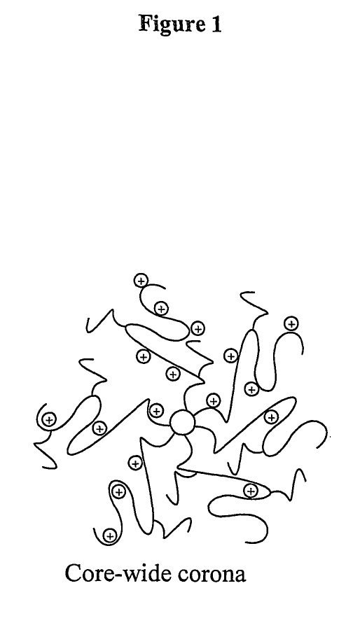

In a first embodiment of the invention, the nanoparticles of the invention are obtainable by emulsion polymerization of at least one water insoluble monomer in an aqueous solution comprising a monomer of formula (I) and a polymer of formula (II). The structure of these nanoparticles is shown schematically in FIG. 1 of the accompanying drawings. The shell forms a corona around the core. The corona structure is able to expand when adsorbing large molecules, such as DNA. The incorporation of the monomer of formula (I) results in the presence of cationic groups on the surface of the nanoparticles which are able to bind nucleic acids to the nanoparticle surface. The incorporation of the polymer of formula (II) results in the presence of poly(ethylene glycol) (PEG) chains in the nanoparticles which produce a highly hydrophilic outer shell.

R¹ in the monomer of formula (I) is hydrogen or methyl, and is preferably methyl.

R² in the monomer of formula (I) may be —COOAOH, —COO—A—NR⁹R¹⁰R¹¹ or —COO—A—N⁺R⁹R¹⁰R¹¹ X⁻ and is preferably —COO—A—NR⁹R¹⁰ or —COO—A—N⁺R⁹R¹⁰R¹¹X⁻.

A in the monomer of formula (I) is C₁₋₂₀ alkylene and is preferably a C₁₋₁₀ alkylene group, more preferably a C₁₋₆ alkylene group, for example a methylene, ethylene, propylene, butylene, pentylene or hexylene group or isomer thereof. Ethylene is preferred.

R⁹ in the monomer of formula (I) is hydrogen or C₁₋₂₀ alkyl, and is preferably a C₁₋₂₀ alkyl group, more preferably a C₁₋₁₀ alkyl group, even more preferably a C₁₋₆ alkyl group, for example a methyl, ethyl, propyl, i-propyl, n-butyl, sec-butyl or tert-butyl group, or a pentyl or hexyl group or isomer thereof. Methyl and ethyl are preferred, particularly methyl.

R¹⁰ in the monomer of formula (I) is hydrogen or C₁₋₂₀ alkyl, and is preferably a C₁₋₂₀ alkyl group, more preferably a C₁₋₁₀ alkyl group, even more preferably a C₁₋₆ alkyl group, for example a methyl, ethyl, propyl, i-propyl, n-butyl, sec-butyl or tert-butyl group, or a pentyl or hexyl group or isomer thereof. Methyl and ethyl are preferred, particularly methyl.

R¹¹ in the monomer of formula (I) is hydrogen or C₁₋₂₀ alkyl, and is preferably a C₁₋₂₀ alkyl group, more preferably a C₄-C₁₆ alkyl group, even more preferably a C₆₋₁₀ alkyl group, for example a hexyl, heptyl, octyl, nonyl or decyl group or isomer thereof. n-Octyl is preferred.

An example of a monomer of formula (I) which may be used in the present invention is 2-(dimethyloctyl) ammonium ethyl methacrylate bromine, which has the formula (1) shown below: 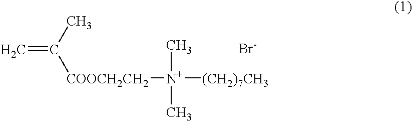
R³ in the polymer of formula (II) is hydrogen or methyl, and is preferably methyl.

R⁴ in the polymer of formula (II) is hydrogen or C₁₋₂₀ alkyl, and is preferably a C₁₋₂₀ alkyl group, more preferably a C₁₋₁₀ alkyl group, even more preferably a C₁₋₆ alkyl group, for example a methyl, ethyl, propyl, i-propyl, n-butyl, sec-butyl or tert-butyl group, or a pentyl or hexyl group or isomer thereof. Methyl and ethyl are preferred, particularly methyl.

n is an integer such that the polymer of formula (II) has a number-average molecular weight of at least 1000. When the number-average molecular weight of the polymer of formula (I) is at least 1000, it is found that the nanoparticles are able to reversibly bind nucleic acids. When the number-average molecular weight is less than 1000, the nanoparticles have a reduced ability to bind eg. plasmid DNA. In view of the DNA binding ability, the number-average molecular weight of the polymer of formula (II) is preferably 1000 to 6000, more preferably 1500 to 3000, and most preferably 1900 to 2100.

An example of a polymer of formula (II) which may be used in the present invention is poly(ethylene glycol) methyl ether methacrylate having a number-average molecular weight of approximately 2000. A suitable polymer is commercially available from Aldrich, and has the formula (2) shown below:
In a second embodiment of the invention, the nanoparticles of the invention are obtainable by 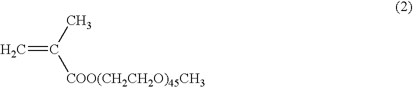
emulsion polymerization of a water insoluble monomer in an aqueous solution comprising a hydrophilic polymer which comprises repeating units of formulae (III) and (IV).

R⁵ in the repeating unit of formula (III) is hydrogen or methyl.

In a particular embodiment R⁶ in the monomer of formula (II) represents hydrogen or —A—NR⁹R¹⁰.

The preferred values of A, R⁹, R¹⁰ and R¹¹ in the repeating unit of formula (III) are the same as described above for the monomer of formula (I).

R⁷ in the repeating unit of formula (IV) is hydrogen or methyl.

R⁸ in the repeating unit of formula (IV) is C₁₋₁₀ alkyl, and is preferably a C₁₋₆ alkyl group, for example a methyl, ethyl, propyl, i-propyl, n-butyl, sec-butyl or tert-butyl group, or a pentyl or hexyl group or isomer thereof. Methyl, ethyl and butyl are preferred.

X in the monomer of formula (I) or repeating unit of formula (III) may be a halogen, sulphate, sulphonate or perchlorate. The halogen may be fluorine, chlorine, bromine or iodine, preferably bromine or iodine, most preferably bromine.

An example of a copolymer comprising repeating units of formulae (III) and (IV) which may be used in the present invention is a copolymer of methacrylic acid and ethyl acrylate, for example a statistical copolymer in which the ratio of the free carboxyl groups to the ester groups is approximately 1:1. A suitable copolymer is commercially available from Röhm Pharma under the trade name Eudragit® L 100-55.

A further example of a copolymer comprising repeating units of formulae (III) and (IV) which may be used in the present invention is a copolymer of 2-(dimethylamino)ethyl methacrylate and C₁₋₆ alkyl methacrylate, for example a copolymer of 2-(dimethylamino)ethyl methacrylate, methyl methacrylate and butyl methacrylate. A suitable copolymer is commercially available from Röhm Pharma under the trade name Eudragit® E 100. The present invention provides a new polymeric delivery system for pharmacologically active substances such as nucleic acids based on polymeric nanoparticles with a core-shell structure and a tailored surface. The inner core is mainly constituted of a water-insoluble polymer or copolymer such as poly(methylmethacrylate) and the hydrophilic outer shell is constituted by hydrosoluble copolymers bearing ionic or ionisable functional groups. For example, the cationic polymers are able to reversibly bind ODNs and DNA. The anionic polymers are able to reversibly bind, protect and deliver basic proteins such as Tat Additionally, the nanoparticles may comprise PEG chain brushes which increase the biocompatibility. It is found that the nanoparticles of the first embodiment of the invention are able to bind relatively high amounts of plasmid PCV₀-tat DNA (5-6% w/w) and to release them with distinct kinetic pathways.

The PEG-based shell in the nanoparticles of the first embodiment of the invention prevents, or at least reduces, the nanoparticle clearance from the body by the phagocytic cells of the reticuloendothelial system (RES). In fact, the capture of foreign nanoparticles is believed to be initially mediated by the adsorption of plasma proteins (opsonins), leading to recognition by the phagocytic cells. The hydrophilicity of the PEG chains located at the nanoparticle surface is responsible for both particle surface steric stabilization and induction of dysopsonic effect, masking the presence of the carriers from the recognition of RES. By avoiding opsonization, polymeric nanoparticles can overcome removal by the mononuclear phagocyte system, thus achieving the goal of having a slow-constant release of drug in the circulation for extended periods of time and improving drug pharmacokinetic performances.

The nanoparticles of the invention are able to reversibly bind and deliver pharmacologically active substances, particularly nucleic acids such as DNA, ODNs and proteins, into cells. Binding on the outer shell is desirable because it prevents degradation of the pharmacologically active substance and allows its release, in the biologically active form, both in vitro and in vivo.

These nanoparticles of the invention are synthesized by emulsion polymerization employing functionalised comonomers as emulsion stabilizers. Emulsion polymerization systems without regular emulsifiers are well known (Gilbert et al., Emulsion Polymerization, A Mechanistic Approach, Academic Press: London, 1995; Wu et al., Macromolecules (1997), 30, 2187; Liu et al., Langmuir (1997), 13, 4988; Schoonbrood et al., Macromolecules (1997), 30, 6024; Cochin et al., Macromolecules (1997), 30, 2287-2287; Xu et al., Langmuir (2001), 17, 6077-6085; Delair et al., Colloid Polym. Sci. (1994), 272, 962), and essentially involve one reactive component, namely “surfmer” or “polymerizable surfactant” which acts to stabilize the emulsion recipe.

As reported in many emulsion polymerization systems including water soluble comonomers (Gilbert et al., Emulsion Polymerization, A Mechanistic Approach, Academic Press: London, 1995; Delair et al., Colloid Polym. Sci. (1994), 272, 962), the complex particle forming mechanism involves homogeneous nucleation. The reaction starts in the aqueous phase leading to the formation of water-soluble oligoradicals, rich in the water soluble comonomer, until they reach the limit of solubility and precipitate to form primary particles which are able to growth by incorporation of the monomer and comonomer. The water soluble units are preferentially located at the nanoparticle surface and actively participate to the latex stabilization. In this way, nanoparticles can be obtained with a tailored surface dictated by the chemical structure of the employed comonomer.

In the emulsion polymerization process to prepare nanoparticles of the present invention the monomers and, if present, polymers are preferably mixed together before emulsion polymerization takes place. This allows production of the core-shell structure of the nanoparticles with the shell forming a corona around the core as shown in FIG. 1.

Specifically, the nanoparticles of the invention may be prepared by emulsion polymerization of a water-insoluble monomer in an aqueous solution comprising:

(i) a monomer of formula (I) and a polymer of formula (II), or

(ii) a hydrophilic copolymer which comprises repeating units of formulae (III) and

The polymerization reaction is typically carried out by introducing the water-insoluble monomer, preferably dropwise, into an aqueous solution comprising the monomer of formula (I) and the polymer of formula (II), or comprising the hydrophilic copolymer which comprises repeating units of formulae (III) and (IV). The reaction is preferably carried out under an inert atmosphere, such as nitrogen, preferably with constant stirring. The aqueous solution may comprise a further solvent, such as acetone. For example a 90/10 vol % water/acetone mixture may be used.

Following addition of the water-insoluble monomer, the system is preferably left to stabilize for a time, e.g. for 10 to 60 minutes, preferably 15 to 40 minutes, prior to addition of a free radical initiator. Examples of suitable free radical initiators include anionic potassium persulfate (KPS), ammonium persulphate and cationic 2,2′-azobis(2-methylpropionamidine) dihydrochloride (AIBA). The free radical initiator is typically added in the form of an aqueous solution.

Polymerization is typically performed at a temperature of 50 to 100° C., preferably 65 to 85° C., for at least 90 minutes. In some cases, the reaction may take as long as 20 hours or more.

At the end of the reaction, the product may be purified by known methods. For example, the product may be filtered and purified by repeated dialysis, e.g. ten times or more against an aqueous solution of cetyl trimethyl ammonium bromide, and then ten times or more against water.

Following the isolation of the nanoparticles from the emulsion, the nanoparticles may be dried by exposure to air or by other conventional drying techniques such as lyophilization, vacuum drying, drying over a desiccant, or the like. Prior to adsorption of a pharmacologically active agent, the nanoparticles may be redispersed in a suitable liquid and temporarily stored. The skilled person will recognise under what conditions the nanoparticles of the invention may be stored. Typically, the nanoparticles are stored at a low temperature, for example about 4□C.

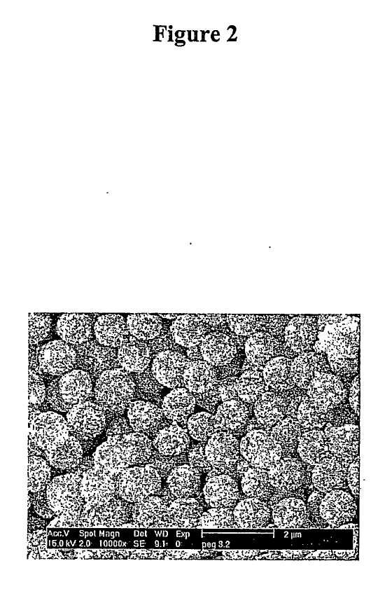

The nanoparticles usually have a spherical shape, although irregularly-shaped nanoparticles are possible. When viewed under a microscope, therefore, the nanoparticles are typically spheroidal but may be elliptical, irregular in shape or toroidal. In certain embodiments the nanoparticles have a raspberry-like morphology, as shown in FIG. 2.

The starting materials of formulae (I), (II), (III) and (IV) are commercially available or may be prepared by known methods. For example, a monomer of formula (I) in which R² represents —A—N⁺R⁹R¹⁰R¹¹X⁻ may be prepared by reacting a compound of formula (VI):
H₂C═C(—R¹)—COO—A—NR⁹R¹⁰ (VI)
with a compound of formula R¹¹X.

The nanoparticles of the invention generally have a number-average particle diameter measured by scanning electron microscopy of less than 1100 nm, preferably 50 to 1000 nm more preferably 50 to 500 nm, e.g. 50 to 300 nm. It is found that the particle diameter is dependent on the free radical initiator that is used during the synthesis of the nanoparticles. For example, samples obtained using AIBA as the free radical initiator generally have a lower number-average particle diameter than samples obtained using KPS. Size reduction is advantageous because it means that a greater surface area is available for adsorption of pharmacologically active substances, thus reducing the amount of polymer required to be administered.

The particle size can be measured using conventional techniques such as microscopic techniques (where particles are sized directly and individually rather than grouped statistically), absorption of gasses, or permeability techniques. If desired, automatic particle-size counters can be used (for example, the Coulter Counter, HIAC Counter, or Gelman Automatic Particle Counter) to ascertain average particle size.

Actual nanoparticle density can be readily ascertained using known quantification techniques such as helium pycnometry and the like. Alternatively, envelope (“tap”) density measurements can be used to assess the density of a particulate composition. Envelope density information is particularly useful in characterizing the density of objects of irregular size and shape. Envelope density, or “bulk density,” is the mass of an object divided by its volume, where the volume includes that of its pores and small cavities. Other, indirect methods are available which correlate to density of individual particles. A number of methods of determining envelope density are known in the art, including wax immersion, mercury displacement, water absorption and apparent specific gravity techniques. A number of suitable devices are also available for determining envelope density, for example, the GeoPyc™ Model 1360, available from the Micromeritics Instrument Corp. The difference between the absolute density and envelope density of a sample pharmaceutical composition provides information about the sample's percentage total porosity and specific pore volume.

Nanoparticle morphology, particularly the shape of a particle, can be readily assessed using standard light or electron microscopy. It is preferred that the particles have a spherical or at least substantially spherical shape. It is also preferred that the particles have an axis ratio of 2 or less, i.e. from 2:1 to 1:1, to avoid the presence of rod- or needle-shaped particles. These same microscopic techniques can also be used to assess the particle surface characteristics, for example, the amount and extent of surface voids or degree of porosity.

The nanoparticles of the invention may also comprise a fluorescent chromophore. For example, yellow-green fluorescent nanoparticles may be obtained by adding the fluorescein-based allylic monomer (3): 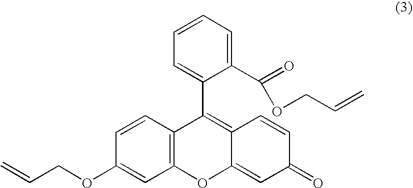
to the polymerization reaction mixture during synthesis of the nanoparticles. The fluorescent monomer (3) is able to polymerize under the employed reaction conditions to give fluorescent nanoparticles. The preparation procedure for these nanoparticles allows the highly fluorescent hydrophobic chromophore to be incorporated into the nanoparticle core. The covalent binding of the dye molecule yields nanoparticles with high fluorescence intensity, minimal quenching and good photostability, so that exposure to light does not reduce their photoemission.

Nanoparticles comprising a fluorescent chromophore may be used as probes in order to get information concerning the core-shell nanoparticle uptake in cellular systems and in vivo.

The nanoparticles of the invention may have pharmacologically-active agent adsorbed at their surface. The term “adsorbed” or “fixed” means that the pharmacologically-active agent is attached to the external surface of the shell of the nanoparticle. The adsorption or fixation preferably occurs by electrostatic attraction. Electrostatic attraction is the attraction or bonding generated between two or more oppositely charged or ionic chemical groups. The adsorption or fixation is typically reversible.

The pharmacologically-active agent preferably has a net charge that attracts it to the ionic or ionisable hydrophilic shell of the nanoparticle. The pharmacologically-active agent typically has one or more charged chemical or ionic groups. In the case of the pharmacologically-active agent being a peptide, the pharmacologically-active agent typically has one or more charged amino acid residues. The pharmacologically-active agent typically has a net positive or negative charge. The pharmacologically-active agent preferably has a net charge that is opposite to the charge of the hydrophilic shell of the nanoparticle.

The pharmacologically-active agent may be adsorbed onto the nanoparticles by mixing a solution of the pharmacologically-active agent with a liquid suspension of the nanoparticles. The pharmacologically-active agent and nanoparticles are typically mixed in a suitable liquid, for example a physiological buffer such as phosphate buffered saline (PBS). The mixture may be left for some time under conditions suitable for the preservation of the pharmacologically-active agent and formation of the bond between the pharmacologically-active agent and nanoparticles. These conditions will be recognised by a person skilled in the art. Adsorption is usually carried out at a temperature of from 0° C. to 37° C., preferably from 4° C. to 25° C. Adsorption may take place in the dark. Adsorption is typically carried out for from 30 and 180 minutes. Following adsorption, the nanoparticles of the invention may be separated from the adsorption liquid by methods known in the art, for example centrifugation. The nanoparticles may then be resuspended in a liquid suitable for administration to an individual.

Pharmacologically-Active Agents Useful in the Invention

A “pharmacologically-active agent” includes any compound or composition of matter which, when administered to an organism (human or animal subject) induces a desired pharmacologic and/or physiologic effect by local and/or systemic action. The term therefore encompasses those compounds or chemicals traditionally regarded as drugs, biopharmaceuticals (including molecules such as peptides, proteins, nucleic acids), vaccines and gene therapies (e.g., gene constructs).

Pharmacologically-active agents useful in this invention include drugs acting at synaptic and neuroeffector junctional sites (cholinergic agonists, anticholinesterase agents, atropine, scopolamine, and related antimuscarinic drugs, catecholamines and sympathomimetic drugs, and adrenergic receptor antagonists); drugs acting on the central nervous systems; autacoids (drug therapy of inflammation); drugs affecting renal function and electrolyte metabolism; cardiovascular drugs; drugs affecting gastrointestinal function; chemotherapy of neoplastic diseases; drugs acting on the blood and the blood-forming organs; and hormones and hormone antagonists. Thus, the agents useful in the invention include, but are not limited to anti-infectives such as antibiotics and antiviral agents; analgesics and analgesic combinations; local and general anesthetics; anorexics; antiarthritics; antiasthmatic agents; anticonvulsants; antidepressants; antihistamines; anti-inflammatory agents; antinauseants; antimigrane agents; antineoplastics; antipruritics; antipsychotics; antipyretics; antispasmodics; cardiovascular preparations (including calcium channel blockers, beta-blockers, beta-agonists and antiarrythmics); antihypertensives; diuretics; vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including double- and single-stranded molecules and supercoiled or condensed molecules, gene constructs, expression vectors, plasmids, antisense molecules and the like.

Specific examples of drugs useful in this invention include angiotensin converting enzyme (ACE) inhibitors, β-lactam antibiotics and γ-aminobutyric acid (GABA)-like compounds. Representative ACE inhibitors are discussed in Goodman and Gilman, Eighth Edition at pp. 757-762, which is incorporated herein by reference. These include quinapril, ramipril, captopril, benzepril, fosinopril, lisinopril, enalapril, and the like and the respective pharmaceutically acceptable salts thereof Beta-lactam antibiotics are those characterized generally by the presence of a beta-lactam ring in the structure of the antibiotic substance and are discussed in Goodman and Gilman, Eighth Edition at pp. 1065 to 1097, which is incorporated herein by reference. These include penicillin and its derivatives such as amoxicillin and cephalosporins. GABA-like compounds may also be found in Goodman and Gilman. Other compounds include calcium channel blockers (e.g., verapamil, nifedipine, nicardipine, nimodipine and diltiazem); bronchodilators such as theophylline; appetite suppressants, such as phenylpropanolamine hydrochloride; antitussives, such as dextromethorphan and its hydrobromide, noscapine, carbetapentane citrate, and chlophedianol hydrochloride; antihistamines, such as terfenadine, phenidamine tartrate, pyrilamine maleate, doxylamine succinate, and phenyltoloxamine citrate; decongestants, such as phenylephrine hydrochloride, phenylpropanolamine hydrochloride, pseudoephedrine hydrochloride, chlorpheniramine hydrochloride, pseudoephedrine hydrochloride, chlorpheniramine maleate, ephedrine, phenylephirine, chlorpheniramine, pyrilamine, phenylpropanolamine, dexchlorpheniramine, phenyltoxamine, phenindamine, oxymetazoline, methscopalamine, pseudoephedrine, brompheniramine, carbinoxamine and their pharmaceutically acceptable salts such as the hydrochloride, maleate, tannate and the like, β-adrenergic receptor antagonists (such as propanolol, nadalol, timolol, pindolol, labetalol, metoprolol, atenolol, esniolol, and acebutolol); narcotic analgesics such as morphine; central nervous system (CNS) stimulants such as methylphenidate hydrochloride; antipsychotics or psychotropics such as phenothiazines, trycyclic antidepressants and MAO inhibitors; benzadiazepines such as alprozolam, diazepam; and the like; and certain non steroidal antinflammatory drugs (NSAIDs), (e.g. salicylates, pyrazolons, indomethacin, sulindac, the fenamates, tolmetin, propionic acid derivatives) such as salicylic acid, aspirin, methyl salicylate, diflunisal, salsalate, phenylbutazone, indomethacin, oxyphenbutazone, apazone, mefenamic acid, meclofenamate sodium, ibuprofen, naproxen, naproxen sodium, fenoprofen, ketoprofen, flurbiprofen, piroxicam, diclofenac, etodolac, ketorolac, aceclofenac, nabumetone, and the like; protease inhibitors, particularly HIV protease inhibitors such as saquinavir, ritonavir, amprenavir, indinavir, lopinavir and nelfinavir.

Another pharmacologically-active agent useful in the invention is an antigen, i.e., molecule which contains one or more epitopes that will stimulate a host's immune system to make a cellular antigen-specific immune response, or a humoral antibody response. Thus, antigens include proteins, polypeptides, antigenic protein fragments, oligosaccharides, polysaccharides, and the like. The antigen can be derived from any known virus, bacterium, parasite, plants, protozoans, or fungus, and can be a whole organism or immunogenic parts thereof, e.g., cell wall components. An antigen can also be derived from a tumor. An oligonucleotide or polynucleotide which expresses an antigen, such as in DNA immunization applications, is also included in the definition of antigen. Synthetic antigens are also included in the definition of antigen, for example, haptens, polyepitopes, flanking epitopes, and other recombinant or recombinant or synthetically derived antigens (Bergmann et al (1993) Eur. J. Immunol. 23:2777-2781; Bergmann et al (1996) J. Immunol. 157:3242-3249; Suhrbier, A. (1997) Immunol. And Cell Biol. 75:402-408; Gardner et al (1998) 12^{th} World AIDS Conference, Geneva, Switzerland (Jun. 28-Jul. 3, 1998).

The antigen is preferably a disease-associated antigen. Thus, a disease-associated antigen is a molecule which contains epitopes that will stimulate a host's immune system to make a cellular antigen-specific immune response, and/or a humoral antibody response against the disease. The disease-associated antigen may therefore be used for prophylactic or therapeutic purposes.

Antigens for use in the invention include, but are not limited to, those containing, or derived from, members of the families Picornaviridae (for example, polioviruses, etc.); Caliciviridae; Togaviridae (for example, rubella virus, dengue virus, etc.); Flaviviridae; Coronaviridae; Reoviridae; Birnaviridae; Rhabodoviridae (for example, rabies virus, measles virus, respiratory syncytial virus, etc.); Orthomyxoviridae (for example, influenza virus types A, B and C, etc.); Bunyaviridae; Arenaviridae; Retroviradae (for example, HTLV-I; HTLV-II; HIV-1; and HIV-2); simian immunodeficiency virus (SIV) among others. Additionally, viral antigens may be derived from a papilloma virus (for example, HPV); a herpes virus, i.e. herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV) and the tick-borne encephalitis viruses; smallpox, parainfluenza, varicella-zoster, cytomeglavirus, Epstein-Barr, rotavirus, rhinovirus, adenovirus, papillomavirus, poliovirus, mumps, rubella, coxsackieviruses, equine encephalitis, Japanese encephalitis, yellow fever, Rift Valley fever, lymphocytic choriomeningitis, and the like. See for example, Virology, 3rd Edition (W. K. Joklik ed. 1988); Fundamental Virology, 2nd Edition (B. N. Fields and D. M. Knipe, eds. 1991), for a description of these and other viruses.

Bacterial antigens include, but are not limited to, those containing or derived from organisms that cause diphtheria, cholera, tuberculosis, tetanus, pertussis, meningitis, and other pathogenic states, including Meningococcus A, B and C, Hemophilus influenza type B (HIB), and Helicobacter pylon, Streptococctus pneumoniae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptspirosis interrogans, Borrelia burgdorferi, Campylobacter jejuni, and the like.

Examples of anti-parasitic antigens include those derived from organisms causing malaria and Lyme disease. Antigens of such fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, Schistosoma mansoni, and the like.

In an especially preferred embodiment, the antigen adsorbed on the nanoparticle is the full length HIV Tat protein or an immunogenic fragment thereof, tat DNA or other DNA or protein which is an HIV antigen. Examples of suitable sequences are given in the sequence listing.

The disease-associated antigen may be cancer-associated. A cancer-associated antigen is a molecule which contains epitopes that will stimulate a host's immune system to make a cellular antigen-specific immune response, and/or a humoral antibody response against the cancer. A cancer-associated antigen is typically found in the body of an individual when that individual has cancer. A cancer-associated antigen is preferably derived from a tumour. Cancer-associated antigens include, but are not limited to, cancer-associated antigens (CAA), for example, CAA-breast, CAA-ovarian and CAA-pancreatic; the melanocyte differentiation antigens, for example, Melan A/MART-1, tyrosinase and gp100; cancer-germ cell (CG) antigens, for example, MAGE and NY-ESO-1; mutational antigens, for example, MUM-1, p53 and CDK4; over-expressed self-antigens, for example, p53 and HER2/NEU and tumour proteins derived from non-primary open reading frame mRNA sequences, for example, LAGE1.

The antigen or immunogenic fragments of antigens mentioned herein typically comprise one or more T cell epitopes. “T cell epitopes” are generally those features of a peptide structure capable of inducing a T cell response. In this regard, it is accepted in the art that T cell epitopes comprise linear peptide determinants that assume extended conformations within the peptide-binding cleft of MHC molecules, (Unanue et al. (1987) Science 236: 551-557). As used herein, a T cell epitope is generally a peptide having about 8-15, preferably 5-10 or more amino acid residues.

The nanoparticles of the invention can be viewed as a “vaccine composition” and as such include any pharmaceutical composition which contains an antigen and which can be used to prevent or treat a disease or condition in a subject. The term encompasses both subunit vaccines, i.e., vaccine compositions containing antigens which are separate and discrete from a whole organism with which the antigen is associated in nature, as well as compositions containing whole killed, attenuated or inactivated bacteria, viruses, parasites or other microbes. The vaccine can also comprise a cytokine that may further improve the effectiveness of the vaccine.

Suitable nucleotide sequences for use in the present invention include any therapeutically relevant nucleotide sequence. Thus, the present invention can be used to deliver one or more genes encoding a protein defective or missing from a target cell genome or one or more genes that encode a non-native protein having a desired biological or therapeutic effect (e.g., an antiviral function) or a sequence that corresponds to a molecule having an antisense or ribozyme function. The invention can also be used to deliver a nucleotide sequence capable of providing immunity, for example an immunogenic sequence that serves to elicit a humoral and/or cellular response in a subject.

Suitable genes which can be delivered include those used for the treatment of inflammatory diseases, autoimmune, chronic and infectious diseases, including such disorders as AIDS, cancer, neurological diseases, cardiovascular disease, hypercholestemia; various blood disorders including various anemias, thalassemia and hemophilia; genetic defects such as cystic fibrosis, Gaucher's Disease, adenosine deaminase (ADA) deficiency, emphysema, etc. A number of antisense oligonucleotides (e.g., short oligonucleotides complementary to sequences around the translational initiation site (AUG codon) of an mRNA) that are useful in antisense therapy for cancer and for viral diseases have been described in the art. See, e.g., Han et al 1991) Proc. Natl. Acad. Sci. USA 88:4313; Uhlmann et al (1990) Chem. Rev. 90:543; Helene et al (1990) Biochim. Biophys. Acta. 1049:99; Agarwal et al (1988) Proc. Natl. Acad. Sci. USA 85:7079; and Heikkila et al (1987) Nature 328:445. A number of ribozymes suitable for use herein have also been described. See, e.g., Chec et al (1992) J. Biol. Chem. 267:17479 and U.S. Pat. No. 5,225,347 to Goldberg et al.

For example, in methods for the treatment of solid tumors, genes encoding toxic peptides (i.e., chemotherapeutic agents such as ricin, diphtheria toxin and cobra venom factor), tumor suppressor genes such as p53, genes coding for mRNA sequences which are antisense to transforming oncogenes, antineoplastic peptides such as tumor necrosis factor (TNF) and other cytokines, or transdominant negative mutants of transforming oncogenes, can be delivered for expression at or near the tumor site.

Similarly, genes coding for peptides known to display antiviral and/or antibacterial activity, or stimulate the host's immune system, can also be administered. Thus, genes encoding many of the various cytokines (or functional fragments thereof), such as the interleukins, interferons and colony stimulating factors, will find use with the instant invention. The gene sequences for a number of these substances are known.

For the treatment of genetic disorders, functional genes corresponding to genes known to be deficient in the particular disorder can be administered to the subject. The instant invention will also find use in antisense therapy, e.g., for the delivery of oligonucleotides able to hybridize to specific complementary sequences thereby inhibiting the transcription and/or translation of these sequences. Thus DNA or RNA coding for proteins necessary for the progress of a particular disease can be targeted, thereby disrupting the disease process. Antisense therapy, and numerous oligonucleotides which are capable of binding specifically and predictably to certain nucleic acid target sequences in order to inhibit or modulate the expression of disease-causing genes are known and readily available to the skilled practitioner. Uhlmann et al (1990) Chem Rev. 90:543, Neckers et al (1992) Crit. Rev. Oncogenesis 3:175; Simons et al (1992) Nature 359:67; Bayever et al (1992) Antisense Res. Dev. 2:109; Whitesell et al (1991) Antisense Res. Dev. 1:343; Cook et al (1991) Anti-cancer Drug Design 6:585; Eguchi et al (1991) Annu. Rev. Biochem. 60:631. Accordingly, antisense oligonucleotides capable of selectively binding to target sequences in host cells are provided herein for use in antisense therapeutics.

The nanoparticles of the invention can comprise from about 0.01 to about 99% of the antigen by weight, for example from about 0.01 to 10%, typically 2 to 8% e.g. 5 to 6% by weight. The actual amount depends on a number of factors including the nature of the pharmacologically-active agent, the dose desired and other variables readily appreciated by those skilled in the art.

When the pharmacologically active agent is an antigen, administration of nanoparticles of the invention generates an immune response in an individual. Adsorption of the antigen to the external surface of the nanoparticle preserves the biological activity of the antigen; adsorption of the antigen to the nanoparticle does not affect the immunogenicity of the antigen. Adsorption of the antigen to the nanoparticle reduces the amount of antigen required to generate an immune response, eliminates or reduces the number of antigen booster shots needed and improves the handling or shelf-life of the antigen.

When the pharmacologically active agent is a drug, biopharmaceutical or gene therapy, administration of nanoparticles of the invention prevents or ameliorates a disease or condition in the man or animal being treated, or assists in the diagnosis of such disease or condition.

Accordingly, the present invention also relates to prophylactic or therapeutic methods utilising the nanoparticles of the invention. When the pharmacologically-active agent is an antigen these prophylactic or therapeutic methods involve generating an immune response in an individual using the nanoparticles of the invention. Thus, the nanoparticles of the invention may be administered to an individual to generate an immune response in that individual. Alternatively, the nanoparticles may be used in the manufacture of a medicament for diagnosing, treating or preventing a condition in an individual particularly generating an immune response in an individual.

The term “administer” or “deliver” is intended to refer to any delivery method of contacting the nanoparticles with the target cells or tissue. The term “tissue” refers to the soft tissues of an animal, patient, subject etc as defined herein, which term includes, but is not limited to, skin, mucosal tissue (eg. buccal, conjunctival, gums), vaginal and the like. Bone may however be treated too by the particles of the invention, for example bone fractures.

When administration is for the purpose of treatment, administration may be either for prophylactic or therapeutic purpose. When provided prophylactically, the pharmacologically-active agent is provided in advance of any symptom. The prophylactic administration of the pharmacologically-active agent serves to prevent or attenuate any subsequent symptom. When provided therapeutically the pharmacologically-active agent is provided at (or shortly after) the onset of a symptom. The therapeutic administration of the pharmacologically-active agent serves to attenuate any actual symptom. Administration and therefore the methods of the invention may be carried out in vivo or in vitro.

The terms “animal”, “individual”, “patient” and “subject” are used interchangeably herein to refer to a subset of organisms which include any member of the subphylum cordata, including, without limitation, humans and other primates, including non-human primates such as chimpanzees and other apes and monkey species; farm animals such as bovine animals, for example cattle; ovine animals, for example sheep; porcine, for example pigs; rabbit, goats and horses; domestic mammals such as dogs and cats; wild animals; laboratory animals including rodents such as mice, rats and guinea pigs; birds, including domestic, wild and game birds such as chickens, turkeys and other gallinaceous birds, ducks, geese; and the like. The terms do not denote a particular age. Thus, both adult and newborn individuals are intended to be covered. In one embodiment, the individual is typically capable of being infected by HIV.

The invention includes a method of diagnosing, treating or preventing a condition in a subject by administering the nanoparticles described herein to a subject in need of such treatment. As used herein, the term “treatment” or “treating” includes any of the following: the prevention of infection or reinfection; the reduction or elimination of symptoms; and the reduction or complete elimination of a pathogen. Treatment may be effected prophylactically (prior to infection) or therapeutically (following infection). The methods of this invention also include effecting a change in an organism by administering the nanoparticles.

The methods of the invention may be carried out on individuals at risk of disease associated with antigen. Typically, the methods of the invention are carried out on individuals at risk of microbial infection or cancer associated with or caused by the antigen. In a preferred embodiment, the method of the invention is carried out on individuals at risk of infection with HIV or developing AIDS.

The methods described herein elicit an immune response against particular antigens for the treatment and/or prevention of a disease and/or any condition which is caused by or exacerbated by the disease. The methods described herein typically elicit an immune response against particular antigens for the treatment and/or prevention of microbial infection or cancer and/or any condition which is caused by or exacerbated by microbial infection or cancer. In a particular embodiment, the methods described herein elicit an immune response against particular antigens for the treatment and/or prevention of HIV infection and/or any condition which is caused by or exacerbated by HIV infection, such as AIDS.

The method of the invention may be carried out for the purpose of stimulating a suitable immune response. By suitable immune response, it is meant that the method can bring about in an immunized subject an immune response characterized by the increased production of antibodies and/or production of B and/or T lymphocytes specific for an antigen, wherein the immune response can protect the subject against subsequent infection. In a preferred embodiment, the method can bring about in an immunized subject an immune response characterized by the increased production of antibodies and/or production of B and/or T lymphocytes specific for HIV-1 Tat, wherein the immune response can protect the subject against subsequent infection with homologous or heterologous strains of HIV, reduce viral burden, bring about resolution of infection in a shorter amount of time relative to a non-immunized subject, or prevent or reduce clinical manifestation of disease symptoms, such as AIDS symptoms.

The aim of this embodiment of the invention is to generate an immune response in an individual. Preferably, antibodies to the antigen are generated in the individual. Preferably IgG, IgA or IgM antibodies to the antigen are generated. Antibody responses may be measured using standard assays such as radioimmunoassay, ELISAs, and the like, well known in the art.

Preferably cell-mediated immunity is generated, and in particular a CD8 T cell response generated. In this case the administration of the nanoparticles may, for example increases the level of antigen experienced CD8 T cells. The CD8 T cell response may be measured using any suitable assay (and thus may be capable of being detected in such an assay), such as an ELISPOT assay, preferably an IFN-γ ELISPOT assay, a CTL assay or peptide proliferation assay. Preferably, a CD4 T cell response is also generated, such as the CD4 Th1 response. Thus the levels of antigen experienced CD4 T cells may also be increased. Such increased levels of CD4 T cells may be detected using a suitable assay, such as a proliferation assay.

The invention further provides the pharmacologically-active nanoparticles of the invention in a pharmaceutical composition which also includes a pharmaceutically acceptable excipient. Such an “excipient” generally refers to a substantially inert material that is nontoxic and does not interact with other components of the composition in a deleterious manner.

These excipients, vehicles and auxiliary substances are generally pharmaceutical agents that do not themselves induce an immune response in the individual receiving the composition, and which may be administered without undue toxicity.

Pharmaceutically acceptable excipients include, but are not limited to, liquids such as water, saline, polyethylene glycol, hyaluronic acid, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like.

It is also preferred, although not required, that a pharmaceutical composition comprising pharmacologically-active nanoparticles will contain a pharmaceutically acceptable carrier that serves as a stabilizer, particularly for peptides, or proteins or the like. Examples of suitable carriers that also act as stabilizers for peptides include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium phosphates, citric acid, tartaric acid, glycine, high molecular weight polyethylene glycols (PEGs), and combination thereof It may also be useful to employ a charged lipid and/or detergent. Suitable charged lipids include, without limitation, phosphatidylcholines (lecithin), and the like. Detergents will typically be a nonionic, anionic, cationic or amphoteric surfactant. Examples of suitable surfactants include, for example, Tergitol® and Triton® surfactants (Union Carbide Chemicals and Plastics, Danbury, Conn.), polyoxyethylenesorbitans, for example, TWEEN® surfactants (Atlas Chemical Industries, Wilmington, Del.), polyoxyethylene ethers, for example Brij, pharmaceutically acceptable fatty acid esters, for example, lauryl sulfate and salts thereof (SDS), and like materials.

A thorough discussion of pharmaceutically acceptable excipients, carriers, stabilizers and other auxiliary substances is available in REMINGTONS PHARMACEUTICAL SCIENCES (Mack Pub. Co., New Jersey 1991), incorporated herein by reference.

In order to augment an immune response in a subject, the compositions and methods described herein can further include ancillary substances/adjuvants, such as pharmacological agents, cytokines, or the like. Suitable adjuvants include any substance that enhances the immune response of the subject to the antigens attached to the nanoparticles of the invention. They may enhance the immune response by affecting any number of pathways, for example, by stabilizing the antigen/MHC complex, by causing more antigen/MHC complex to be present on the cell surface, by enhancing maturation of APCs, or by prolonging the life of APCs (e.g., inhibiting apoptosis).

Typically adjuvants are co-administered with the vaccine or nanoparticle. As used herein the term “adjuvant” refers to any material that enhances the action of a antigen or the like.

Thus, one example of an adjuvant is a “cytokine.” As used herein, the term “cytokine” refers to any one of the numerous factors that exert a variety of effects on cells, for example, inducing growth, proliferation or maturation. Certain cytokines, for example TRANCE, flt-3L, and CD40L, enhance the immunostimulatory capacity of APCs. Non-limiting examples of cytokines which may be used alone or in combination include, interleukin-2 (IL-2), stem cell factor (SCF), interleukin 3 (IL-3), interleukin 6 (IL-6), interleukin 12 (IL-12), G-CSF, granulocyte macrophage-colony stimulating factor (GM-CSF), interleukin-1 alpha (IL-1 a), interleukin-11 (IL-11), MIP-1a, leukemia inhibitory factor (LIF), c-kit ligand, thrombopoietin (TPO), CD40 ligand (CD40L), tumor necrosis factor-related activation-induced cytokine (TRANCE) and flt3 ligand (flt-3L). Cytokines are commercially available from several vendors such as, for example, Genzyme (Framingham, Mass.), Genentech (South San Francisco, Calif.), Amgen (Thousand Oaks, Calif.), R & D Systems and Immunex (Seattle, Wash.).

The sequence of many of these molecules are also available, for example, from the GenBank database. It is intended, although not always explicitly stated, that molecules having similar biological activity as wild-type or purified cytokines (for example, recombinantly produced or mutants thereof) and nucleic acid encoding these molecules are intended to be used within the spirit and scope of the invention.

A composition which contains the nanoparticles of the invention and an adjuvant, or a vaccine or nanoparticles of the invention which is co-administered with an adjuvant, displays “enhanced immunogenicity” when it possesses a greater capacity to elicit an immune response than the immune response elicited by an equivalent amount of the vaccine administered without the adjuvant. Such enhanced immunogenicity can be determined by administering the adjuvant composition and nanoparticle controls to animals and comparing antibody titres and/or cellular-mediated immunity between the two using standard assays such as radioimmunoassay, ELISAs, CTL assays, and the like, well known in the art.

The pharmacologically active nanoparticles may function as an adjuvant. For example they may enhance the immune response when administered with an antigen, compared to administration of the antigen alone. Thus the nanoparticles in this embodiment may be administered separately, simultaneously or sequentially with the antigen.

In the method of the invention the nanoparticles of the invention are typically delivered in liquid form or delivered in powdered form. Liquids containing the nanoparticles of the invention are typically suspensions. The nanoparticles of the invention may be administered in a liquid acceptable for delivery into an individual. Typically the liquid is a sterile buffer, for example sterile phosphate-buffered saline (PBS).

The nanoparticles of the invention are typically delivered parenterally, either subcutaneously, intravenously, intramuscularly, intrasternally or by infusion techniques. A physician will be able to determine the required route of administration for each particular patient.

The vaccine or nanoparticles are typically delivered transdermally. The term “transdermal” delivery intends intradermal (for example, into the dermis or epidermis), transdermal (for example, “percutaneous”) and transmucosal administration, for example, delivery by passage of an agent into or through skin or mucosal (for example buccal, conjunctival or gum) tissue. See, for example, Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987).

Delivery may be via conventional needle and syringe for the liquid suspensions containing nanoparticle particulate. In addition, various liquid jet injectors are known in the art and may be employed to administer the nanoparticles. Methods of determining the most effective means and dosages of administration are well known to those of skill in the art and will vary with the delivery vehicle, the composition of the therapy, the target cells, and the subject being treated. Single and multiple administrations can be carried out with the dose level and pattern being selected by the attending physician. The liquid compositions are administered to the subject to be treated in a manner compatible with the dosage formulation, and in an amount that will be prophylactically and/or therapeutically effective.

The nanoparticles themselves in particulate composition (for example, powder) can also be delivered transdermally to vertebrate tissue using a suitable transdermal particle delivery technique. Various particle delivery devices suitable for administering the substance of interest are known in the art, and will find use in the practice of the invention. A transdermal particle delivery system typically employs a needleless syringe to fire solid particles in controlled doses into and through intact skin and tissue. Various particle delivery devices suitable for particle-mediated delivery techniques are known in the art, and are all suited for use in the practice of the invention. Current device design& employ an explosive, electric or gaseous discharge to propel the coated core carrier particles toward target cells. The coated particles can themselves be releasably attached to a movable carrier sheet, or removably attached to a surface along which a gas stream passes, lifting the particles from the surface and accelerating them toward the target. See, for example, U.S. Pat. No. 5,630,796 which describes a needleless syringe. Other needleless syringe configurations are known in the art.

Delivery of particles from such particle delivery devices is practiced with particles having an approximate size generally ranging from 0.05 to 250 μm. The actual distance which the delivered particles will penetrate a target surface depends upon particle size (e.g., the nominal particle diameter assuming a roughly spherical particle geometry), particle density, the initial velocity at which the particle impacts the surface, and the density and kinematic viscosity of the targeted skin tissue. In this regard, optimal particle densities for use in needleless injection generally range between about 0.1 and 25 g/cm³, preferably between about 0.9 and 1.5 g/cm³, and injection velocities generally range between about 100 and 3,000 m/sec, or greater. With appropriate gas pressure, particles can be accelerated through the nozzle at velocities approaching the supersonic speeds of a driving gas flow.

The powdered compositions are administered to the subject to be treated in a manner compatible with the dosage formulation, and in an amount that will be prophylactically and/or therapeutically effective.

The pharmacologically-active nanoparticles described herein can be delivered in a therapeutically effective amount to any suitable target tissue via the above-described particle delivery devices. For example, the compositions can be delivered to muscle, skin, brain, lung, liver, spleen, bone marrow, thymus, heart, lymph, blood, bone cartilage, pancreas, kidney, gall bladder, stomach, intestine, testis, ovary, uterus, rectum, nervous system, eye, gland and connective tissues.

A “therapeutically effective amount” is defined very broadly as that amount needed to give the desired biologic or pharmacologic effect. This amount will vary with the relative activity of the pharmacologically-active agent to be delivered and can be readily determined through clinical testing based on known activities of the pharmacologically-active agent being delivered. The “Physicians Desk Reference” and “Goodman and Gilman's The Pharmacological Basis of Therapeutics” are useful for the purpose of determined the amount needed in the case of known pharmaceutical agents. The amount of nanoparticles administered depends on the organism (for example animal species), pharmacologically-active agent, route of administration, length of time of treatment and, in the case of animals, the weight, age and health of the animal. One skilled in the art is well aware of the dosages required to treat a particular animal with a pharmacologically-active agent.

Commonly, the nanoparticles are administered in milligram amounts, eg 1 μg to 5 mg, more typically 1 to 50 μg of pharmacologically-active-agent. An appropriate effective amount can be readily determined by one of skill in the art upon reading the instant specification.

Mixed populations of different types of nanoparticles can be combined into single dosage forms and can be co-administered. For example the nanoparticles may have different pharmacologically active agents adsorbed to them. The same pharmacologically-active agent can be incorporated into the different nanoparticle types that are combined in the final formulation or co-administered. Thus, multiphasic delivery of the same pharmacologically-active agent can be achieved.

Below are Examples of specific embodiments for carrying out the present invention. The examples are offered for illustrative purposes only, and are not intended to limit the scope of the present invention in any way.

### EXAMPLES

In the Examples, 2-(dimethylamino)ethyl methacrylate (DMAEMA), 1-bromooctane, poly(ethylene glycol) methyl ether methacrylate (Mₙ=2080) (2), 2,2′-azobis(2-methylpropionamidine) dihydrochloride (AIBA), fluorescein and allyl chloride were purchased from Aldrich. Potassium persulfate (KPS) was purchased from Carlo Erba. The poly(methacrylic acid, ethyl acrylate) 1:1 statistical copolymer (trade name Eudragit® L 100-55) characterized by a number average molecular weight Mₙ of 250000 and the poly(butylmethacrylate, 2-dimethylamino ethyl methacrylate, methyl methacrylate) 1:2:1 statistical copolymer (trade name Eudragit E100) characterized by a number average molecular weight Mn of 150,000, were kindly supplied by Röhm Pharma.

All these products were used without further purification. Methyl methacrylate (MMA) was purchased from Aldrich and distilled under vacuum just before use.

The potentiometric titrations were conducted with a bench pH meter CyberScan pH 1000 equipped with an ATC probe and an Ingold Ag 4805-S7/120 combination silver electrode. The quaternary ammonium group amount per gram of nanoparticle was determined by potentiometric titration of the bromine ions obtained after complete ionic exchange. The ionic exchange was accomplished by dispersing in a beaker 0.5 g of the nanoparticle sample in 25 ml of 1M KNO₃ at room temperature for 48 h. In these conditions, a quantitative ionic exchange was achieved. The mixture was then adjusted to pH=2 with dilute H₂SO₄ and the bromide ions in solution were titrated with a 0.01 M solution of AgNO₃.

The nanoparticle size was measured by a JEOL JSM-35CF scanning electron microscope (SEM) with an accelerating voltage of 10-30 kV. The samples were sputter coated under vacuum with a thin layer (10-30 Å) of gold. The magnification is given by the scale on each micrograph. The SEM photographs were digitalized, using the Kodak photo-CD system, and elaborated by the NIH Image (version 1.55, public domain) image processing program. From 150 to 200 individual nanoparticle diameters were measured for each optical micrograph.

Z-average particle size and polydispersity index (PI) were determined by dynamic light scattering (DLS) at 25° C. with a Zetasizer 3000 HS (Malvern, U.K.) system using a 10 mV He—Ne laser and PCS software for Windows (version 1.34, Malvern, U.K.). For the data analysis, the viscosity and refractive index of pure water at 25° C. were used. The instrument was checked with a standard polystyrene latex with a diameter of 200 nm.

ζ-potential was measured at a temperature of 25° C. with a Zetasizer 3000 HS (Malvern, U.K.) and PCS software for Windows (version 1.34, Malvern, U.K.). The instrument was checked using a latexes with a known ζ-potential.

A schematic representation of the structure of a core-shell nanoparticle obtainable by emulsion polymerization of water insoluble monomer in an aqueous solution comprising a monomer of formula (I) and a polymer of formula (II) is shown in FIG. 1.

### Reference Example 1

Synthesis of Ionic Monomer (1)

The ionic monomer 2-(dimethyloctyl)ammonium ethyl methacrylate bromine (1) was obtained by direct reaction of DMAEMA with 1-bromooctane. DMAEMA (0.166 mol) was mixed with 1-bromooctane (0.083 mol) without any additional solvent. After the addition of a small portion of hydroquinone to inhibit eventual radical polymerization reactions, the mixture was stirred at 50° C. for 24 h. The solid product so obtained was washed with dry diethyl ether to remove the excess DMAEMA. Finally, it was dried under vacuum at room temperature. The purity of the product was tested by ¹H NMR spectra. Reaction yields were in the 55-65% range.

### Reference Example 2

Synthesis of Fluorescent Monomer (3)

2.0 g of fluorescein (6.0 mmol), 2.0 g of calcium carbonate and hydroquinone (trace) were dissolved in 100 ml of DMF, and the solution was heated at 60° C. Allyl chloride was added slowly dropwise and the reaction was allowed to proceed for 30 h in the dark. After vacuum evaporation of the solvent the product was purified by flash column chromatography (silica gel; diethyl ether-ethyl acetate 80:20 as eluent). Yield 53%, (m.p.=123-125° C.); MS, m/z (%): 412 (M+, 100), 371 (10), 287 (20), 259 (15), 202 (7); ¹H-NMR (CD₃OD): d 4.44 (dd, J=5.9 and 1 Hz, 2 H, O—CH₂—CH═), 4.75 (dd, J=5.9 and 1 Hz, 2 H, O—CH₂—CH═), 5.08 (m, 2H, CH₂═CH), 5.40 (m, 2H, CH₂═CH), 5.58 (m, 1H, CH₂═CH), 6.10 (m, 1H, CH₂═CH), 6.60 (m, 2H, Ar), 6.98 (m, 3H, Ar), 7.25 (d, J=1 Hz, 1H, Ar), 7.45 (dd, J=7.5 and 1 Hz, 1H, Ar), 7.85 (m, 2H, Ar), 8.30 (dd, J=7.5 and 1 Hz, 1H, Ar).

### Example 1

Nanoparticle Preparation

In a typical emulsion polymerization reaction, 6.0 ml (56.2 mmol) of methyl methacrylate were introduced in a flask containing 120 ml of an aqueous solution of the ionic monomer (1) obtained in Reference Example 1 and non-ionic polymer (2). The flask was fluxed with nitrogen under constant stirring for 30 min, then anionic KPS or cationic AIBA dissolved in water were added. The final amounts of initiator and comonomers in the various sample are listed in Table 1.

The flask was fluxed with nitrogen during the polymerization which was performed at 80±1.0° C. for 24 hours under constant stirring. At the end of the reaction, the product was filtered and purified by repeated dialysis, at least ten times, against an aqueous solution of cetyl trimethyl ammonium bromide, to remove the residual methyl methacrylate, and then water, at least ten times, to remove the residual comonomer. The nanoparticle yield, with respect to the total amount of methyl methacrylate and of the water-soluble comonomers, was comprised between 50 and 60%.

It was found that the emulsion polymerization reaction of methyl methacrylate in the presence of specifically designed reactive surfactants and comonomers leads to monodisperse nanoparticles with a core-shell structure and a tailored surface. The inner core is mainly constituted of poly(methyl methacrylate). At the end of the reaction the water soluble units are covalently bound at the nanoparticle surface and actively participate to the latex stabilization. In this way, nanoparticles can be obtained with a tailored surface dictated by the chemical structure of the employed comonomer.

These nanoparticles were prepared by emulsion polymerization employing methyl methacrylate as the monomer, and two water-soluble comonomers, the ionic monomer (1), bearing a positively charged ammonium group, and the non-ionic polymer (2), bearing a PEG chain with number average molecular weight Mₙ=2080. Table 1 reports the composition of polymerisation reaction mixture, whereas Table 2 reports the physical characteristics of the obtained samples. Unexpectedly, these samples presented a raspberry-like morphologies, as shown in FIG. 2.

To clarify the influence of the free radical initiator on the nanoparticle characteristics, in particular on the size, two different initiators were used namely, anionic KPS and cationic AIBA. Sample obtained with AIBA as free radical sources shown a mean diameter definitely lower with respect to samples obtained with KPS.

TABLE 1
Composition of emulsion polymerization reaction mixture (total
volume = 126 ml)

| | Comonomer | | | | Reaction |
|---|---|---|---|---|---|
| | 1 | Polymer 2 | Comonomer 3 | Initiator | time |
| Sample | (mmol) | (mmol) | (μmol) | (mmol) | (hours) |
| PEG1 | 3.00 | 0.14 | | KPS | 2 |
| | | | | 0.22 | |
| PEG2 | 3.00 | 0.31 | | KPS | 2 |
| | | | | 0.22 | |
| PEG3 | 3.00 | 0.52 | | KPS | 2 |
| | | | | 0.22 | |
| PEG3 | 12.50 | 2.16 | 48.0 | KPS | 2 |
| fluo* | | | | 0.92 | |
| PEG4 | 3.00 | 0.70 | | KPS | 2 |
| | | | | 0.22 | |
| PEG32* | 12.50 | 2.16 | | KPS | 2 |
| | | | | 0.92 | |
| Z2 | 3.60 | 0.52 | | AIBA | 4 |
| | | | | 0.092 | |
| Z2fluo* | 15.0 | 2.13 | 18.9 | AIBA | 4 |
| | | | | 0.38 | |
| Z3 | 3.60 | 0.78 | | AIBA | 4 |
| | | | | 0.092 | |

*large scale synthesis (total volume = 500 ml)

TABLE 2
Nanoparticle physico-chemical characterization

| | SEM | | | Surface |
|---|---|---|---|---|
| | diameter | PCS diameter | Zp | charge density |
| Sample | (nm) | (nm) | (mV) | (μmol m⁻²) |
| PEG1 | 930 ± 290 | / | / | 27.5 |
| PEG2 | 890 ± 140 | / | / | 2.24 |
| PEG3 | 550 ± 200 | 469.0 ± 3.5 | +34.7 ± 0.3 | 6.66 |
| PEG3 fluo | 627 ± 38 | 663.8 ± 38.09 | +16.6 ± 0.6 | 10.9 |
| PEG4 | 460 ± 60 | / | / | 2.08 |
| PEG32 | 960 ± 38 | 923.6 ± 3.9 | +32.2 ± 0.6 | 2.16 |
| Z2 | 180 ± 18 | 218 ± 60 | +17.7 ± 1.2 | 6.35 |
| Z3 | 136 ± 13 | 160 ± 61 | +9.9 ± 1.2 | 2.63 |
| Z2fluo | / | 204.3 ± 0.5 | +17.6 ± 0.9 | / |

### Example 2

Nanoparticles Preparation

Table 3 reports the composition of polymerisation reaction mixture. Table 4 reports the physical characteristics of the obtained samples.

TABLE 3
Composition of emulsion polymerization reaction
mixture (total volume = 500 ml)

| | Ionic monomer 1 | Polymer 2 | AIBA |
|---|---|---|---|
| Sample | (mmol) | (mmol) | (mmol) |
| ZP1 | 10.0 | 0.26 | 0.313 |
| ZP2 | 10.0 | 0.78 | 0.313 |
| ZP3 | 10.0 | 1.56 | 0.313 |
| ZP4 | 10.0 | 3.30 | 0.313 |

TABLE 4
Nanoparticle physico-chemical characterization

| | | | | Surface charge |
|---|---|---|---|---|
| | SEM dimeter | PCS diameter | Zp | density (BR + C1) |
| Sample | (nM) | (nm) | (mV) | (μmol/g) |
| ZP1 | 244 ± 63 | 238.2 ± 2.2 | 37.3 ± 0.7 | 100 |
| ZP2 | 212 ± 22 | 235.5 ± 1.0 | 24.5 ± 1.8 | 91 |
| ZP3 | 200 ± 29 | 257.4 ± 3.2 | 18.3 ± 1.0 | 80 |
| ZP4 | / | 219.1 ± 1.2 | 10.4 ± 0.3 | 32 |

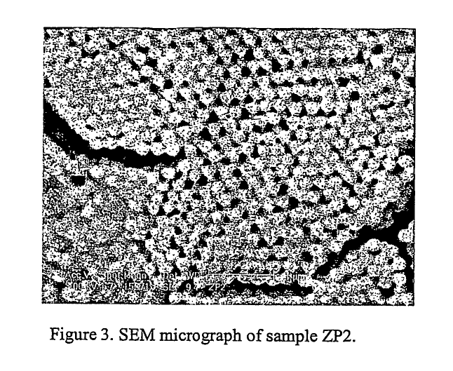

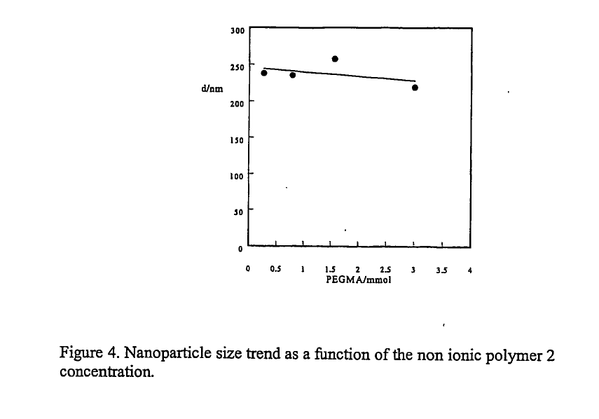

Table 4 reports some physicochemical characteristics of the obtained nanoparticles. As a typical example, FIG. 3 illustrates the SEM image of sample ZP2, whereas FIG. 4 illustrates the diameter trend estimated by PCS as a function of the non-ionic polymer 2 concentration.

The nanoparticle samples presents average diameters ranging from 220 to 260 nm for series. In all cases, a very narrow size distribution was obtained and the nanoparticle size decreases regularly as the non ionic polymer 2 concentration increases (FIG. 4).

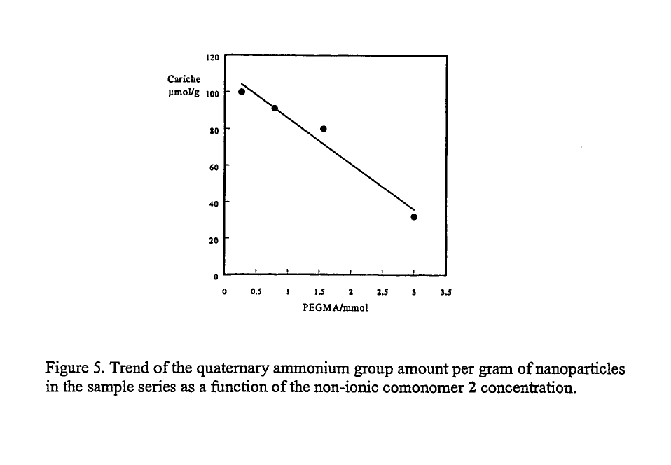

The amount of the ionic comonomer units per gram of nanoparticles was estimated from the titration data. FIG. 5 illustrates the trend of the quaternary ammonium group amount per gram in the sample series as a function of the non-ionic comonomer 2 concentration. Along each series, the quaternary ammonium group amount per gram of nanoparticles decreases linearly with increasing comonomer 2 concentration.

### Example 3

Nanoparticle Preparation

In a typical emulsion polymerization reaction, the appropriate amount of Eudragit® L100-55 was introduced in a flask containing 200 ml of water or a mixture water/acetone 90/10 vol % (see Table 5) adjusted at pH 8.0 with NaOH. The flask was fluxed with nitrogen under constant stirring then 25.0 ml (234 mmol) of MMA were added dropwise. The system was let to stabilize for 20 min, then 21.0 mg (77.7 μmol) of KPS dissolved in 2 ml of water were added. The polymerization was performed at 70±1.0° C. for 17 h. At the end of the reaction, the product was filtered and purified by repeated dialysis against water. The nanoparticle yield, with respect to the methyl methacrylate was comprised between 75 and 90%. A fluorescent nanoparticle sample was prepared in a large scale synthesis: 7.5 g of Eudragit® L100-55 was introduced in a 1L five-neck reactor containing 500 ml of water (see Table 5) adjusted at pH 8.0 with NaOH. The reactor was fluxed with nitrogen under constant stirring then 39 mg of the fluorescent monomer (3) obtained in Reference Example 2 dissolved in 62.0 ml (580 mmol) of MMA were added dropwise. The system was let to stabilize for 20 min, then 52.5 mg (194 μmol) of KPS dissolved in 3 ml of water were added. The polymerization was performed at 70±1.0° C. for 17 h. At the end of the reaction, the product was purified as previously described.

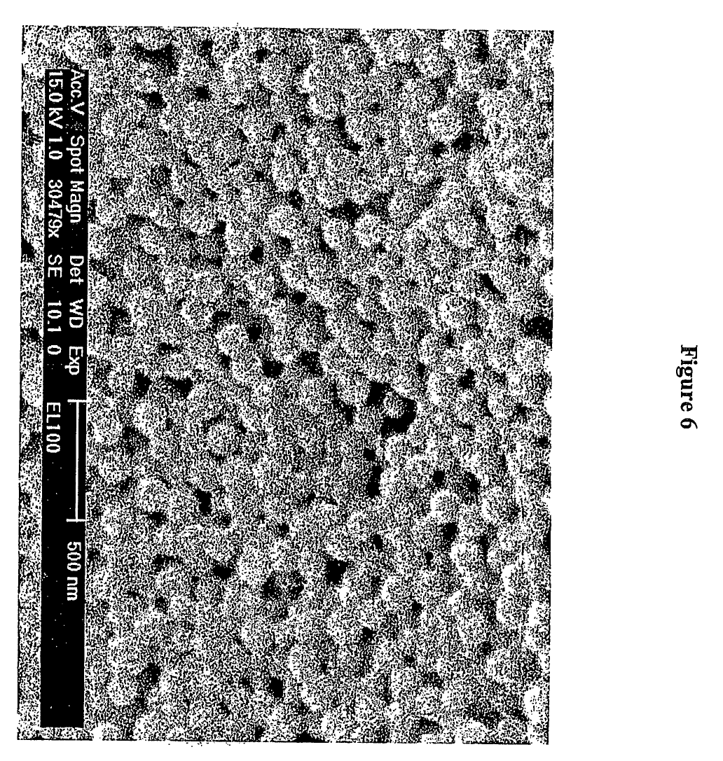

As a typical example, a SEM micrograph of sample M1 is reported in FIG. 6 whereas Table 5 collects some physicochemical characteristics of the samples including the number average diameter calculated by SEM and PCS. In addition, the ζ ζ-potential values are reported. The size of the nanoparticles is small, ranging from 120 to 140 nm. The size of the nanoparticles increases in water, as can be observed from the comparison of the diameters from SEM and PCS, due to the presence of the Eudragit® L 100/55 at the surface in agreement with their core-shell nature. This result is also supported by the negative ζ ζ-potential values due to the presence of negatively charged carboxylic groups of the stabilizer.

Thus polymethylmethacrylate core-shell particles in the nanometre scale range can be prepared by emulsion polymerization. The nature of the outer layer is dictated by the stabilizer Eudragit® L 100/55 which affords:

i) steric stabilization to the latex;

ii) a hydrophilic outer layer deriving able to decrease the particle capture by RES and to influence the particle biodistribution; and

iii) carboxyl groups able to interact with Tat via specific or non specific interactions.

TABLE 5
Amount of monomer (MMA), and stabilizer (Eudragit ® L100-55),
reaction medium composition, number average diameter as determined by
SEM analysis (D_{SEM}), average diameter as determined by PCS
analysis (D_{PCS}), and ζ ζ-potential for samples M1-M4.

| | MMA | Eudragit ® | Reaction | D_{SEM} | D_{PCS} | ζ-potential |
|---|---|---|---|---|---|---|
| Sample | Mmol | L100-55 g | medium | nm | nm | mV |
| M1 | 234.0 | 1.00 | water | 136 | 197 | −45.4 |
| M2 | 234.0 | 2.00 | water | 128 | 188 | −45 |
| M3 | 234.0 | 3.00 | water | 136 | 182 | −46 |
| M4 | 234.0 | 2.00 | water/ | 140 | 213 | −45.7 |
| | | | acetone | | | |
| | | | 90/10 vol % | | | |
| M2 fluo | 580.0 | 7.4 | water | / | 154 | −52.9 |

### Example 4

In a typical emulsion polymerization reaction, 2.0 g of Eudragit E 100 were introduced in a 1L five-neck reactor containing 500 ml of water adjusted at pH 3.0 with HCl. The reactor was fluxed with nitrogen under constant stirring then 75.0 ml (702 mmol) of MMA were added dropwise. The system was let to stabilize for 20 min, then 62.0 mg (229 μmol) of KPS dissolved in 3 ml of water were added. The polymerization was performed at 70±1.0° C. for 17 h. At the end of the reaction, the product was purified as described in example 3. Sample MC3 was obtained with a diameter of 67 nm.

### Example 5

Nanoparticle Preparation

In a typical emulsion polymerisation reaction, 2.0 g of Eudragit L100-55 or Eudragit E 100 were introduced in a flask containing 500 ml of water (see Table 6) and adjusted at pH 8.0 with NaOH in the case of Eudragit L100-55 or pH 2 with hydrochloric acid in the case of Eudragit E 100. The flask was fluxed with nitrogen under constant stirring then the appropriate amount of MMA (see Table 6) was added dropwise. The system was let to stabilize for 20 min, then 62.0 mg of KPS dissolved in 2 ml of water were added. The polymerization was performed at 80±1.0° C. for 4 h. At the end of the reaction, the product was filtered and purified by repeated dialysis, at least ten times, against water. The nanoparticles yield, with respect to the methyl methacrylate was comprised between 75 and 90%.

TABLE 6
Physicochemical characteristics of nanoparticles MAn and MCn.

| Sample | [MMA]/M | diameter/nm | Sample | [MMA]/M | diameter/nm |
|---|---|---|---|---|---|
| MA8 | 0.0934 | 84 | MC8 | 0.0934 | 26 |
| MA9 | 0.28 | 128 | MC9 | 0.28 | 35 |
| MA1 | 0.467 | 143 | MC1 | 0.467 | 49 |
| MA2 | 0.934 | 198 | MC2 | 0.934 | 66 |
| MA3 | 1.402 | 246 | MC3 | 1.402 | 65 |
| MA4 | 1.968 | 250 | MC4 | 1.968 | 98 |
| MA5 | 2.335 | 257 | MC5 | 2.335 | 72 |
| MA6 | 2.804 | 272 | MC6 | 2.804 | 86 |
| MA7 | 3.372 | 289 | MC7 | 3.372 | 95 |

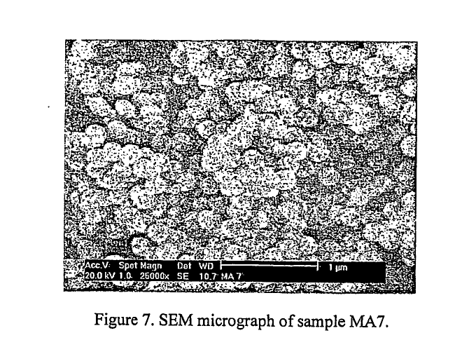

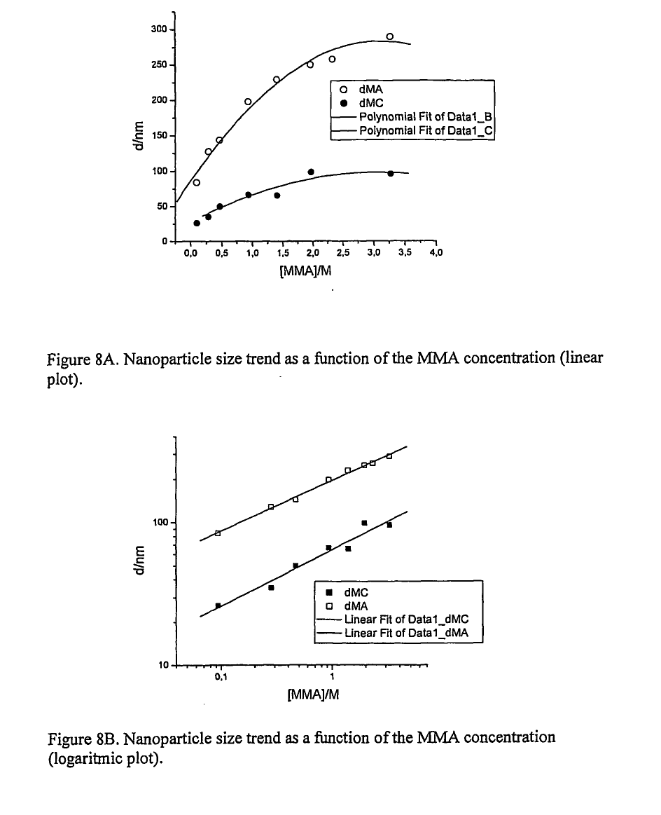

Table 6 reports some physicochemical characteristics of the obtained nanoparticles. As a typical example, FIG. 7 illustrates the SEM image of sample MA7. FIGS. 8A and 8B illustrate the diameter trend estimated by PCS of both sample series as a function of the MMA concentration.

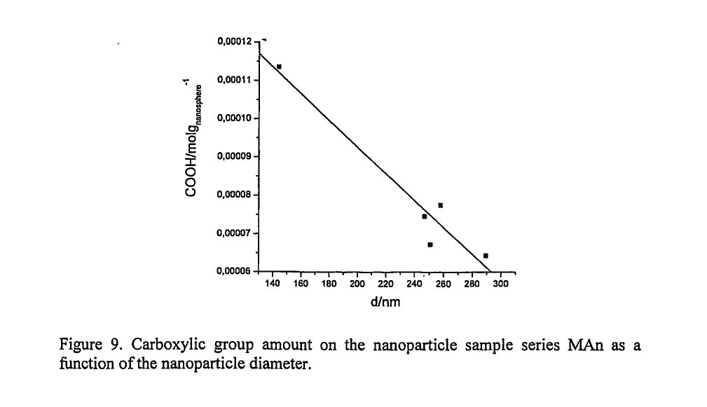

The nanoparticle samples have average diameters ranging from 84 to 289 nm for series MAn and from 26 to 98 nm for series MCn. In all cases, a very narrow size distribution was obtained and, for both series, the nanoparticles size increase regularly as the MMA concentration increases (FIG. 8A). In addition, a linear size vs. MMA concentration relationship was obtained using a logarithmic scale in which the fit lines result nearly parallel each other. This implies that the nanoparticle size is connected to the MMA concentration through a power low with very similar power low coefficients which result 0.360 for series MCn and 0.355 for series MAn. This allows the nanoparticle size to be predetermined according to the initial MMA concentration. In the view of the special interest for nanoparticles MAn as TAT delivery systems, additional characteristics were studied for this sample series. For some samples belonging to series MAn, the carboxylic group amount was also determined by back titration and its trend, as a function of the nanoparticle size is illustrated in FIG. 9.

The amount of carboxylic group on the nanoparticles decreases regularly as the nanoparticles diameter increases, thus suggesting a constant carboxylic group surface density in the various samples. To get information concering the surface characteristics of the nanoparticles samples, the Z-potential of sample MA7 was determined at different pH values. The ζ-potential decreases steeply at first and then more gradually as the pH increases till a limiting value of −45 mV is reached at pH greater than 6, in agreement with the complete dissociation of the carboxylic groups. This indicates that the nanoparticles surface at physiological pH is able to interact through electrostatic interactions with positively charged proteins and in particular with TAT protein.

### Examples 6 to 8

In these examples, binding—release experiments in cell-free systems were carried out using the following nanoparticles whose preparation has been described above:

### Positively Charged Nanoparticles for DNA Delivery (PEG 2000)

| | SEM | PCS | ζ-potential | Surface charge |
|---|---|---|---|---|
| Sample | (nm) | (nm) | (mV) | (μmol/g) |
| PEG32 | 960 | 923.6 | +32.2 | 18 |
| ZP3 | 200 | 257.4 | +18.3 | 80 |

### Acid Nanoparticles for Protein Delivery (Eudragit L100-55)

| | SEM | PCS | ζ-potential | Surface charge |
|---|---|---|---|---|
| Sample | (μm) | (μm) | (mV) | (μmol/g) |
| MA7 | 219.5 | 289 | −50.1 | 64.3 |

### Example 6

ODN/DNA Adsorption Experiments on Pegylated Nanoparticles

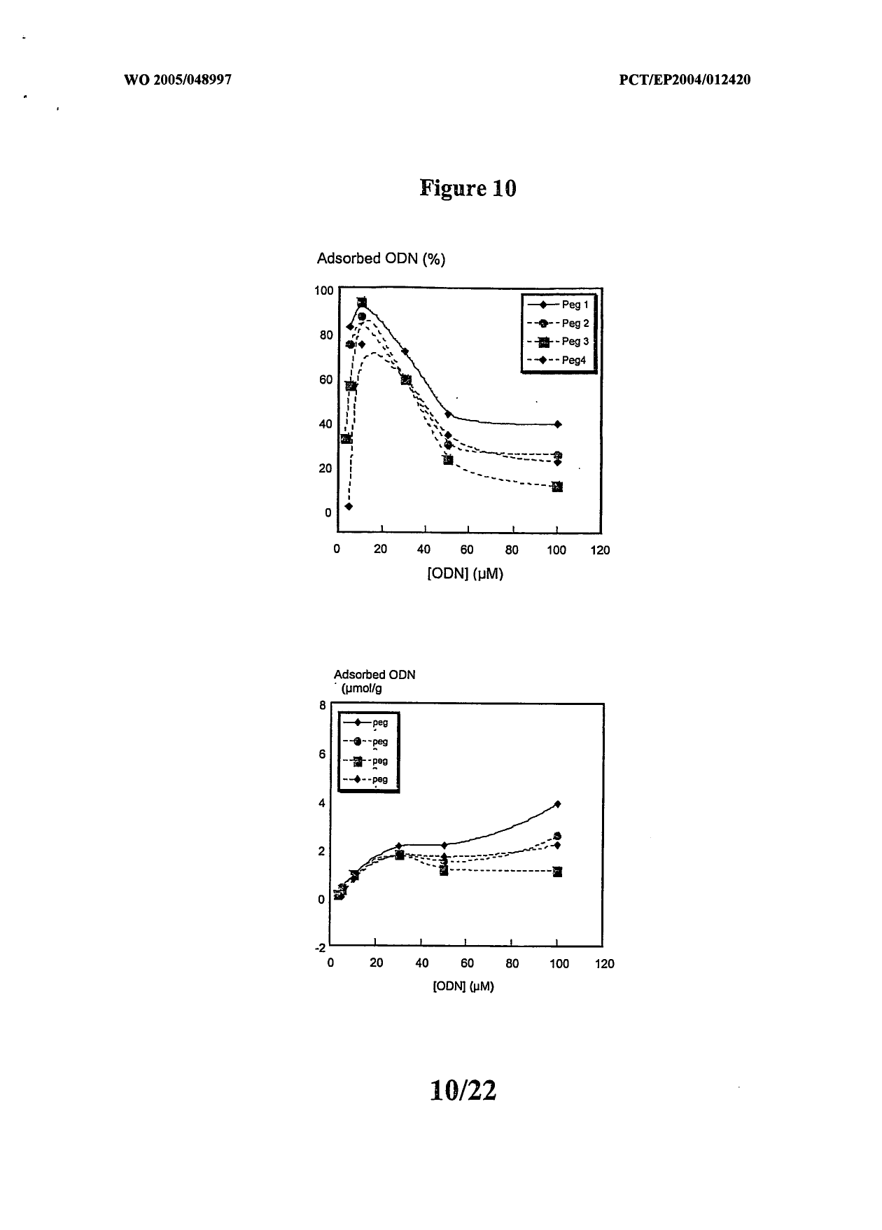

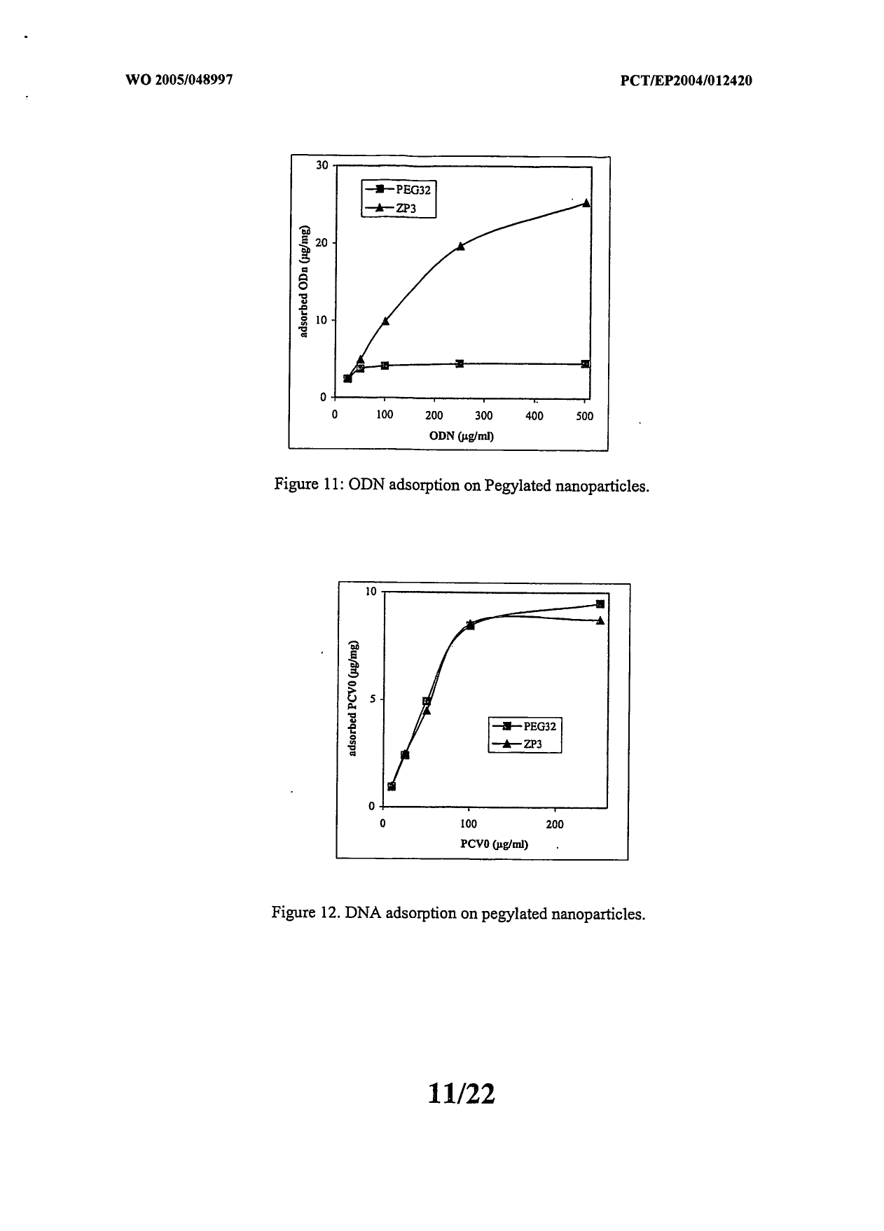

For ODN adsorption experiments, 5.0 mg of freeze-dried nanoparticles were suspended in 0.5 ml of 20 mM sodium phosphate buffer (pH 7.4) and sonicated for 15 min. The appropriate amount of a concentrated aqueous solution of ODN was then added to reach the final concentration (10-200 μM). Several oligomers (18 mer to 22 mer) were tested and the interaction with the nanoparticles was found to be not sequence specific. The experiments were run in triplicate (SD≦10%). The suspensions were continuously stirred at 25° C. for 2 h. After centrifugation at about 9000 rpm for 5 min, quantitative sedimentation of the ODN-nanoparticle complex was obtained and aliquots (10-50 μl) of the supernatant were withdrawn, filtered on a Millex GV₄ filter unit and diluted with sodium phosphate buffer. Finally, UV absorbance at λ=260 nm was measured. Adsorption efficiency (%) was calculated as 100×(administered ODN)−(unbound ODN)/(administered ODN). Adsorption experiments in the presence of model oligo(deoxy) nucleotides (ODN) showed a similar behaviour for PEG1, PEG2, PEG3 and PEG4 (FIG. 10). ODN adsorption on ZP3 and PEG32 samples is shown in FIG. 11.

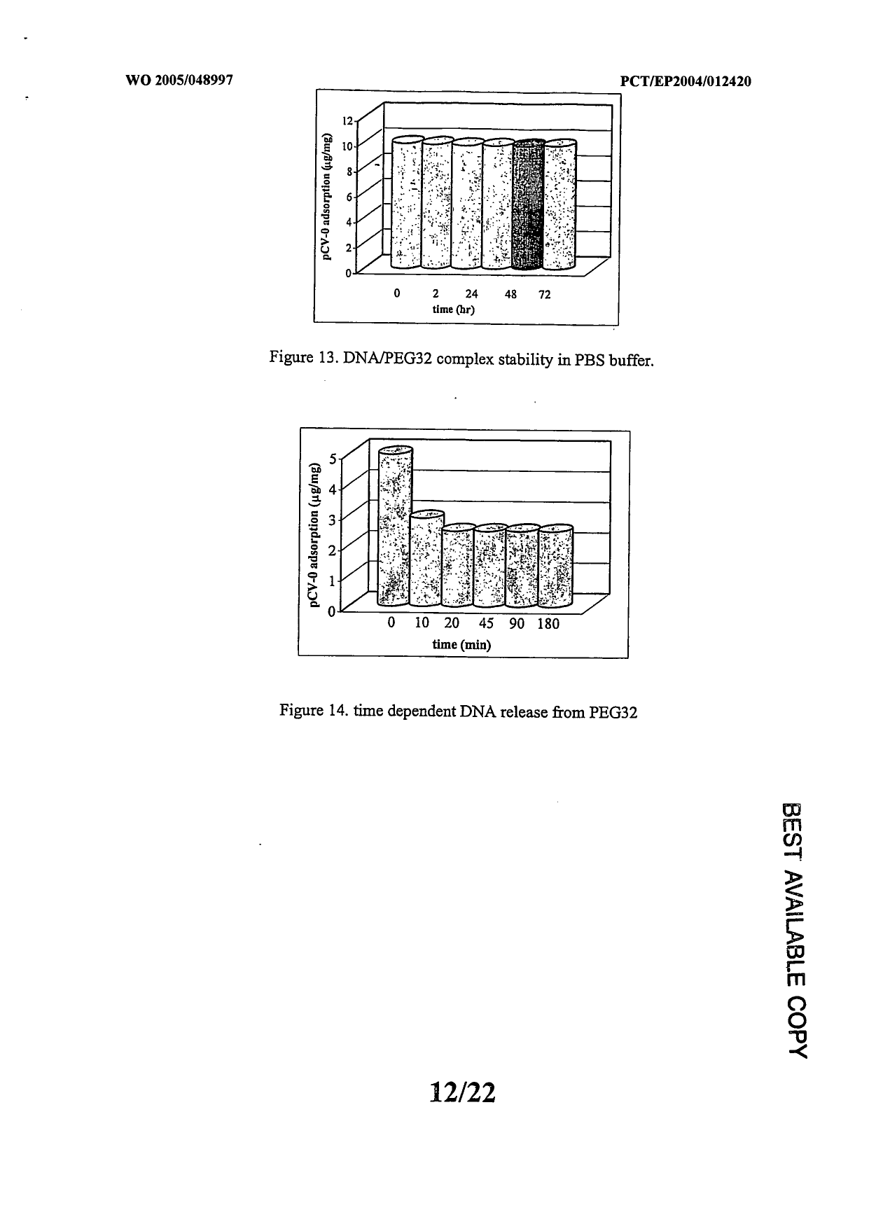

Similarly, DNA adsorption experiments were run by adding the appropriate amount of a concentrated aqueous solution of DNA to reach the final concentration (10-250 μg/ml). Again adsorption of plasmid DNA was found to be not sequence specific. As shown in FIG. 12, pCV0 plasmid DNA can be quantitatively adsorbed on pegylated particle surface, when given in concentration up to 100 μg/ml. PEG32 and ZP3 nanoparticles are able to bind relatively high amounts of plasmid pCV—tat DNA. DNA/PEG32 complexes are stable in physiological buffers (FIG. 13).

### Example 7

ODN/DNA Release Experiments on Pegylated Nanoparticles

The nanoparticle samples (5.0 mg/0.5 ml) were charged with the appropriate amount of ODN and DNA. After 2 hours at room temperature under stirring and centrifugation, the pellet was washed twice with buffer. Release was monitored after 2 h at room temperature in the presence of various NaCl concentration phosphate buffers (20 mM, pH 7.4) through direct measurement of released DNA absorbance (λ=260 nm). The experiments were run in triplicate (SD≦10%). ODN interaction with PEG32 surface is reversible: after 2 hours at room temperature in 1M NaCl phosphate buffer (pH 7.4), ODN release from PEG32 nanoparticles is extensive (87%), whereas approximately up to 50% of bound DNA is quickly released under the same experimental conditions. FIG. 14 shows the release of DNA from PEG 32 nanoparticles over time in the presence of high salted phosphate buffer (pH 7.4).

### Example 8

Protein Adsorption/Release on Nanoparticles

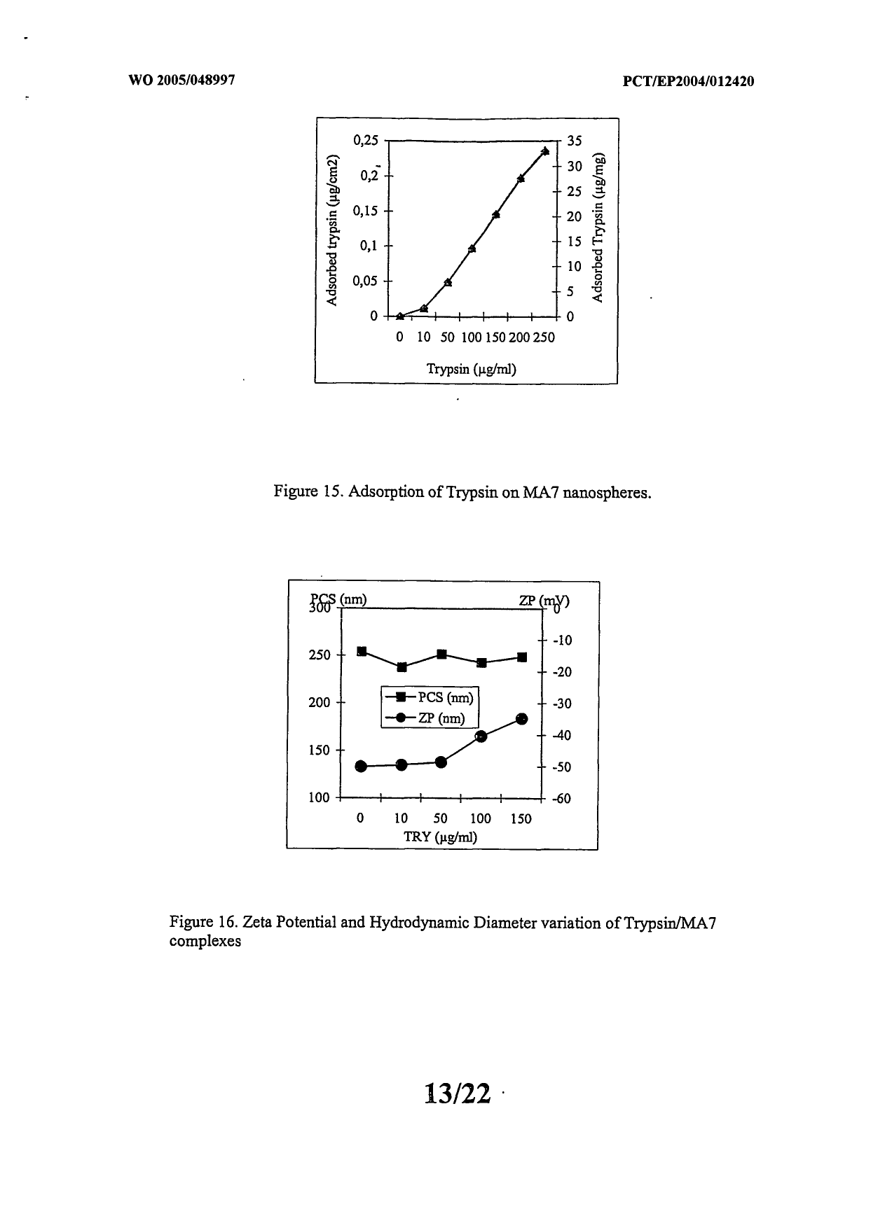

Increasing amounts of proteins were added to the nanoparticle suspension (5 mg/ml) in 10 mM phosphate buffer (pH 7.4) at room temperature. The samples were incubated at room temperature for 2 hours under continuous stirring. After centrifugation at 14000 rpm for 10 minutes, supernatants were filtrated (0.2 μm) and diluted with phosphate buffer before UV absorbance detection at 280 nm or through colorimetric tests (Bradford). Experiments were run in triplicate. (SD<10). Extensive trypsin adsorption occurs with high efficiency rates as shown in FIG. 15.

The effect of a model basic protein (i.e. Trypsin) adsorption on MA7 acid nanoparticles surface in water was studied by means of dynamic light scattering techniques. Binding of small amounts of proteins does not affect the particle hydrodynamic diameter size, whereas it promotes a significative reduction in ζ-potential values as expected by a partial neutralization of the surface carboxylic groups upon protein binding. FIG. 16 shows how PCS and zeta-potential varies with binding of trypsin (TRY) on MA7 nanoparticles.

### Examples 9 to 13 and Reference Example 3

### PEG3 and PEG32

In these examples, in vitro and in vivo experiments were carried out with the following nanoparticles, whose preparation has been described before, to assess their ability to act as a delivery system for DNA vaccination:

Physical properties of polymeric core-shell nanoparticles^{a}

| | SEM | PCS | | Surface charge |
|---|---|---|---|---|
| Nano- | diameter | diameter | ζ-potential | density |
| particle | (nm) | (nm) | (mV) | (μmol NR₄⁺ m⁻²) |
| PEG3 | 550 ± 200 | 470 ± 3.5 | +34.7 ± 0.3 | 6.66 |
| PEG32 | 960 ± 38 | 923 ± 3.9 | +32.2 ± 0.6 | 2.16 |
| PEG3-fluo | 627 ± 38 | 663.8 ± 38 | +16.6 ± 0.6 | 10.9 |

^{a}Physical properties of polymeric core-shell nanoparticles composed of an inner hard core made of poly(methylamino)ethyl methacrylate surrounded by an outer shell of poly(ethylene)glycol chain brushes with functional positive charged groups. Nanoparticles were synthetized as described in materials and methods.

Plasmids

Plasmid pCV-tat, expressing the HIV-1 tat cDNA (HLTV-III, BH10 clone) under the transcriptional control of the adenovirus major late promoter and the empty plasmid pCV-0 has been described by Arya S. K. et al., Science 229:69-73, 1985. Plasmid pGL2-CMV-Luc-basic expressing the luciferase gene cDNA, under the transcriptional control of the human cytomegalovirus, was purchased from Promega (Milan, Italy). Plasmid DNAs were purified onto two CsCl gradients, and resuspended in sterile phosphate-buffered saline (PBS), without calcium and magnesium, according to standard procedures.

Cells Cultures

Monolayer cultures of HeLa and HL3T1 cells, the latter containing an integrated copy of plasmid HIV-1-LTR-CAT, where expression of the chloramphenicol acetyl transferase (CAT) reporter gene is driven by the HIV-1 LTR promoter, were obtained through the American Type Cell culture collection (ATCC) and grown in DMEM (Gibco, Grand Island, N.Y.) containing 10% FBS (Hyclone, Logan, Utah) (Wright C M, et al., Science 234:988-92, 1986). BALB/c 3T3 and BALB/c 3T3-Tat murine fibroblasts (aplotype H^{2kd}), stably transfected with plasmid pRP-neo-c, or with pRP-neo-Tat, respectively, were described by Caputo et al., J. Acquir, Immune Defic. Syndr. 3:372-379, 1990 and grown in DMEM supplemented with 10% FBS. P815 cells (aplotype H^{2kd}) derived from a murine mastocytome were obtained through ATCC and grown in RPMI 1640 (Gibco) containing 10% FBS.

### Example 9

Cell-Free Adsorption/Release Experiments

To assess DNA adsorption onto particles surface, freeze-dried nanoparticles were suspended (10 mg/ml) in 20 mM sodium phosphate buffer (pH 7.4) in a volume of 500 μl, and stirred for 5 minutes. Increasing amounts of pCV-0 plasmid DNA (10-250 μg/ml) were then added. The suspensions were continuously stirred for 2 hours at room temperature. After centrifugation at 9000 rpm for 15 minutes, the supernatants were collected, filtered through Filtek RC4 filter unit (0.2 μm, Chemtek, Germany), and UV absorbance was measured at 260 nm to determine the amount of unbound DNA. Adsorption efficiency (%) was calculated as 100×[(administered DNA)−(unbound DNA)/(administered DNA)]. The experiments were run in triplicate (SD≦10%).

For DNA release experiments, DNA/PEG3 nanoparticle complexes were prepared using the ratio of 25 μg of DNA/mg of PEG3 nanoparticles/ml of 20 mM sodium phosphate buffer (pH 7.4). DNA/PEG32 nanoparticle complexes were prepared using 10 and 100 μg of pCV-0 plasmid DNA/10 mg of PEG32/ml of 20 mM sodium phosphate buffer (pH 7.4). After 2 hours incubation at room temperature, the PEG3/ and PEG32/DNA complexes were collected by centrifugation, resuspended in the same volume, used for complex assembly, of 1 M NaCl/20 mM sodium phosphate buffer (pH 7.4), and incubated at 37° C. under continuous stirring. At different time intervals, samples were spun at 9000 rpm for 15 minutes and supernatants analysed by agarose gel electrophoresis to determine the amount of DNA released from the complexes. DNA quantification was carried out using a densitometer gel analyzer (Quantity-One, BioRad Laboratories, Milan, Italy) as compared to known amounts of plasmid DNA migrated in each gel. Percentage (%) of DNA released from the complexes was determined as 100×(released DNA/bound DNA). The experiments were run in triplicate (SD≦10%).

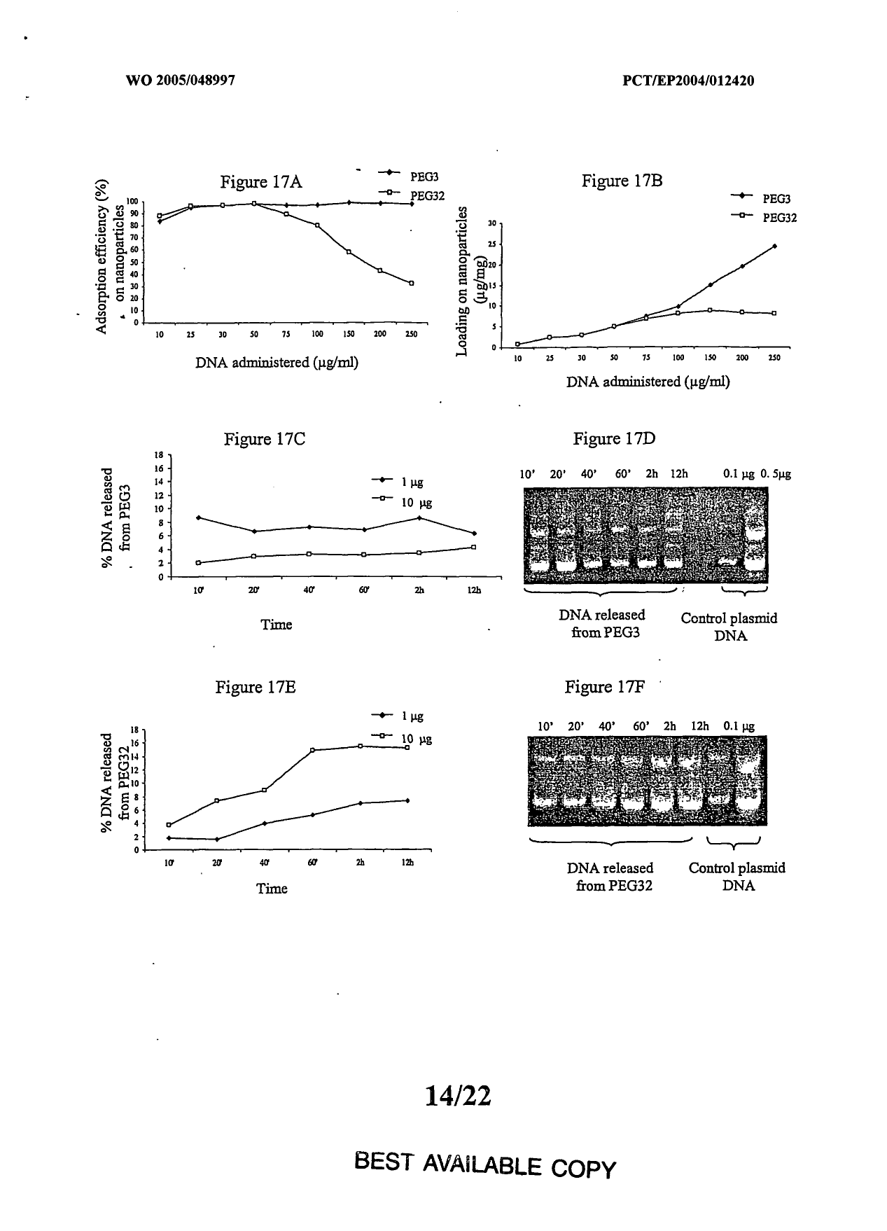

The adsorption trend is shown in FIG. 17A (adsorption efficiency) and in FIG. 17B (DNA loading). Sample PEG3 showed the highest DNA binding ability (up to 25 μg/mg) together with the highest adsorption efficiency, at least in the concentration range 10-250 μg/ml. Conversely, for sample PEG32, surface saturation occurred at lower DNA concentration (100 μg/ml), leading to lower loading values (≅8 μg/mg). The different adsorption behavior between PEG3 and PEG32 correlates with the difference in surface charge density, indicating that adsorption is mainly driven by electrostatic interaction between the negative charges of DNA molecules and the positive charges of the core-shell nanoparticles surface.

To assess whether the DNA adsorbed onto the nanoparticle surface is then released, PEG3/and PEG32/DNA complexes were incubated at 37° C. for different time periods in the presence of 1 M NaCl/20 mM phosphate buffer (pH 7.4). After incubation, complexes were centrifuged and the DNA, released in each supernatant, was analyzed by agarose gel electrophoresis. As shown in FIGS. 17C (PEG3 complexes) and 17E (PEG32 complexes), in the presence of a high salt concentration the DNA is released in a time-dependent fashion from PEG3 and PEG32. This result confirms that electrostatic interactions represent the major driving force for DNA adsorption and release on/from these core-shell nanoparticles. However, the kinetics of DNA release appear different between PEG3 and PEG32. In particular, the efficiency of DNA release from PEG32 nanoparticles increases with time at both doses of 1 and 10 μg, and seems to be related to the amount of bound DNA, being greater from samples loaded with 10 μg. Finally, the results of these experiments showed that the DNA released both from PEG3 (FIG. 17D) and PEG32 (FIG. 17F) nanoparticles preserved its structural integrity. Indeed, the ratio between super-coiled and coiled plasmid DNA conformation remained unchanged, as compared to control plasmid DNA. In conclusion, these results demonstrate that the DNA is efficiently adsorbed and released on/from the particles surface and that it is not degraded or damaged during the adsorption and release processes.

### Example 10

Analysis of Cytotoxicity in Vitro

HL3T1 cells (1×10⁴/100 μl) were seeded in 96-well plates and cultured at 37° C. for 24 hours. Medium was then replaced with 100 μl of medium containing increasing concentrations of PEG3 (20-400 μg/ml) and PEG32 (50-500 μg/ml) nanoparticles. Each sample was assayed in sextupled wells. Cells were incubated for 96 hours at 37° C., and cell proliferation was measured using the colorimetric cell proliferation kit I (MTT based) (Roche, Milan, Italy)

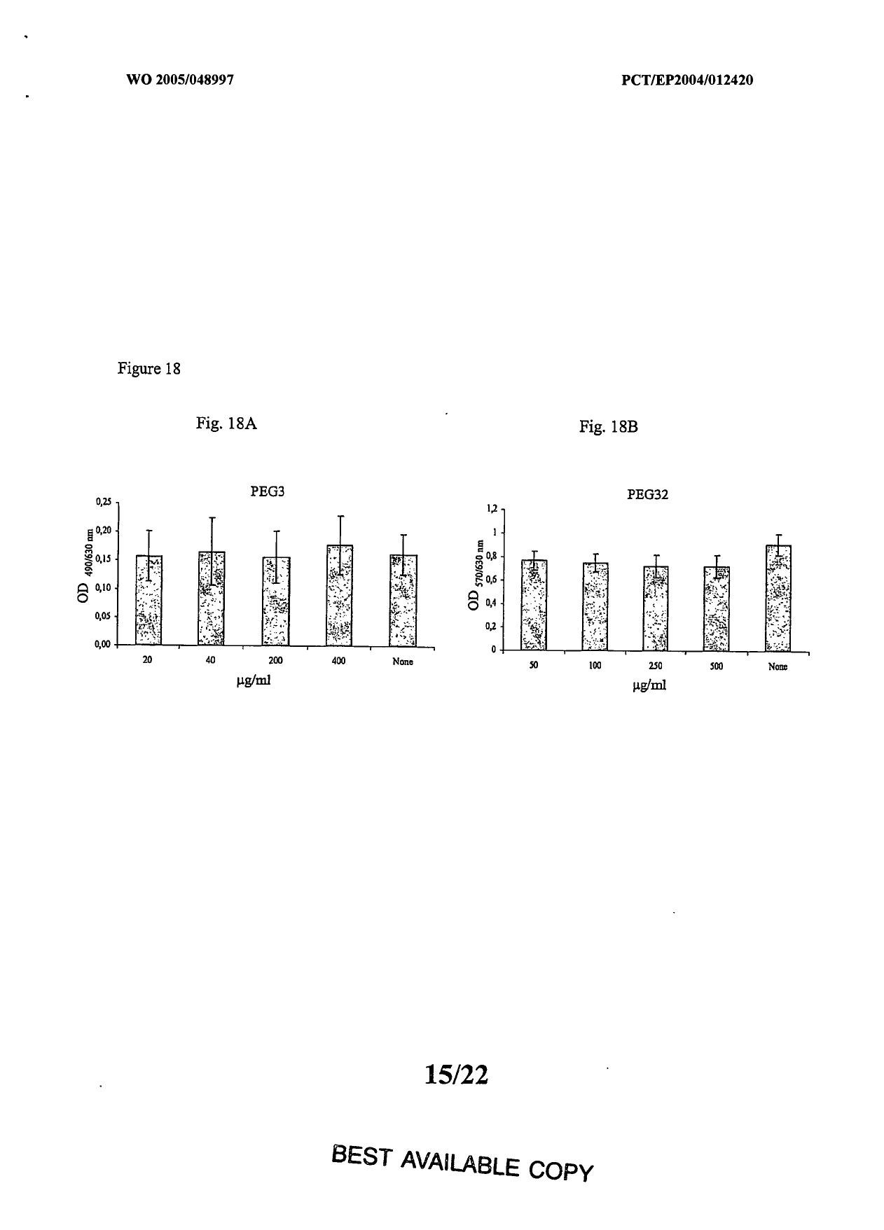

As shown in FIG. 18, no significant reduction (p>0.05) of cell viability was observed after 96 hours incubation in the samples treated both with PEG3 and PEG32, as compared to untreated cells. Similar results were obtained with DNA/nanoparticle complexes (data not shown).

### Example 11

Cellular Uptake

The internalization of the pCV-0 DNA/nanoparticle complexes from the cells was assessed by using PEG3-fluo nanoparticles. HL3T1 cells (5×10⁴/well) were seeded in 24-well plates containing 12-mm coverslips and cultured at 37° C. Twenty-four hours later, pCV-0/PEG3-fluo complexes, prepared at the ratio of 25 μg/mg/ml, as described in Example 9 and resuspended in 200 μl of DMEM containing 10% FBS, were added to the cells. Controls were represented by untreated cells and cells incubated with PEG3-fluo unloaded nanoparticles. At different time intervals, cells were washed with PBS, fixed with 4% cold paraformaldehyde and observed at a confocal laser scanning microscope LSM410 (Zeiss, Oberkochen, Germany). Image acquisition, recording and filtering were carried out using a Indy 4400 graphic workstation (Silicon Graphics, Mountain View, Calif.) as described in eg. Betti et al., Vaccine 19: 3408-3419, 2001.

Seven-weeks old female BDF mice (n=3) were injected with 1 mg of PEG-fluo nanoparticles resuspended in 100 μl of PBS in the quadriceps muscle of the left posterior leg. Mice were injected with 100 μl of PBS alone, as control, in the quadriceps muscle of the right posterior leg. Fifteen and 30 minutes after injection mice were anesthetized intraperitoneally with 100 μl of isotonic solution containing 1 mg of Inoketan (Virbac, Milan, Italy), and 200 μg Rompun (Bayer, Milan, Italy), and sacrificed. Muscle samples at the site of injections were removed, immediately submerged in liquid nitrogen for 1 minute and stored at −80° C. Five μm frozen sections were prepared, fixed with fresh 4% paraformaldehyde for 10 minutes at room temperature, washed with PBS, and colored with DAPI (0.5 μg/ml; Sigma) for 10 minutes, which stains the nuclei. After one wash with PBS, the sections were dried with ethanol, mounted in glycerol/PBS containing 1,4-diazabicyclo[2.2.2]octane to retard fading, and observed at a fluorescence microscope (Axiophot 100, Zeiss). The green fluorescence (microspheres) was observed with a 450-490 λ, flow through 510 λ and long pass 520 λ filter; the blue fluorescence (DAPI) was observed with a band pass 365 λ, flow through 395 λ and long pass 397 λ filter. For the same microscopic field, green and blue images were taken with a Cool-Snapp CCD camera (DAPI) was observed with a band pass 365 λ, flow through 395 λ and long pass 397 λ filter. For the same microscopic field, green and blue images were taken with a Cool-Snapp CCD camera (RS-Photometrics, Fairfax, Va.). The images were then overlapped using the Adobe Photoshop 5.5 program.

Thus, to asses the capability of the nanoparticles to be taken up by cells, a fluorescent core-shell nanoparticle sample, namely PEG3-fluo, was prepared. Since fluorescent particles obtained by simple dye adsorption at their surface can give rise to desorption of the dye and loss of fluorescent emission, following exposure to light and during in vitro experiments, the sample was prepared using a reactive fluoresceine derivative (monomer 3). Although allylic monomers do not undergo radical polymerization, they are able to co-polymerize or at least to be included in the polymer chain as a terminal group. Accordingly, the nanoparticle sample PEG3-fluo was prepared by running the emulsion polymerization reaction in the same experimental conditions as sample PEG3 with the addition of a small amount of the fluorescent monomer 3. A nanoparticle sample with an average diameter, determined by SEM microscopy, of 627±38 nm and a surface charge density of 10.9 μmol m⁻² was obtained (Table 2). This sample presents an emission maximum at 535 nm (λ_{exc}=488 nm) and good photo-stability. After the conventional purification procedure, a small amount of PEG3-fluo was dissolved in chloroform and precipitated in methanol. The polymeric material appeared fluorescent, whereas no trace of fluorescence was observed in the precipitation medium, thus demonstrating that the fluorescent units, deriving from monomer 3, were covalently bound to the PMMA constituting the inner core of the nanoparticles. In addition, the intensity of the fluorescence of the nanoparticles exposed to light for 30 days remained unchanged.

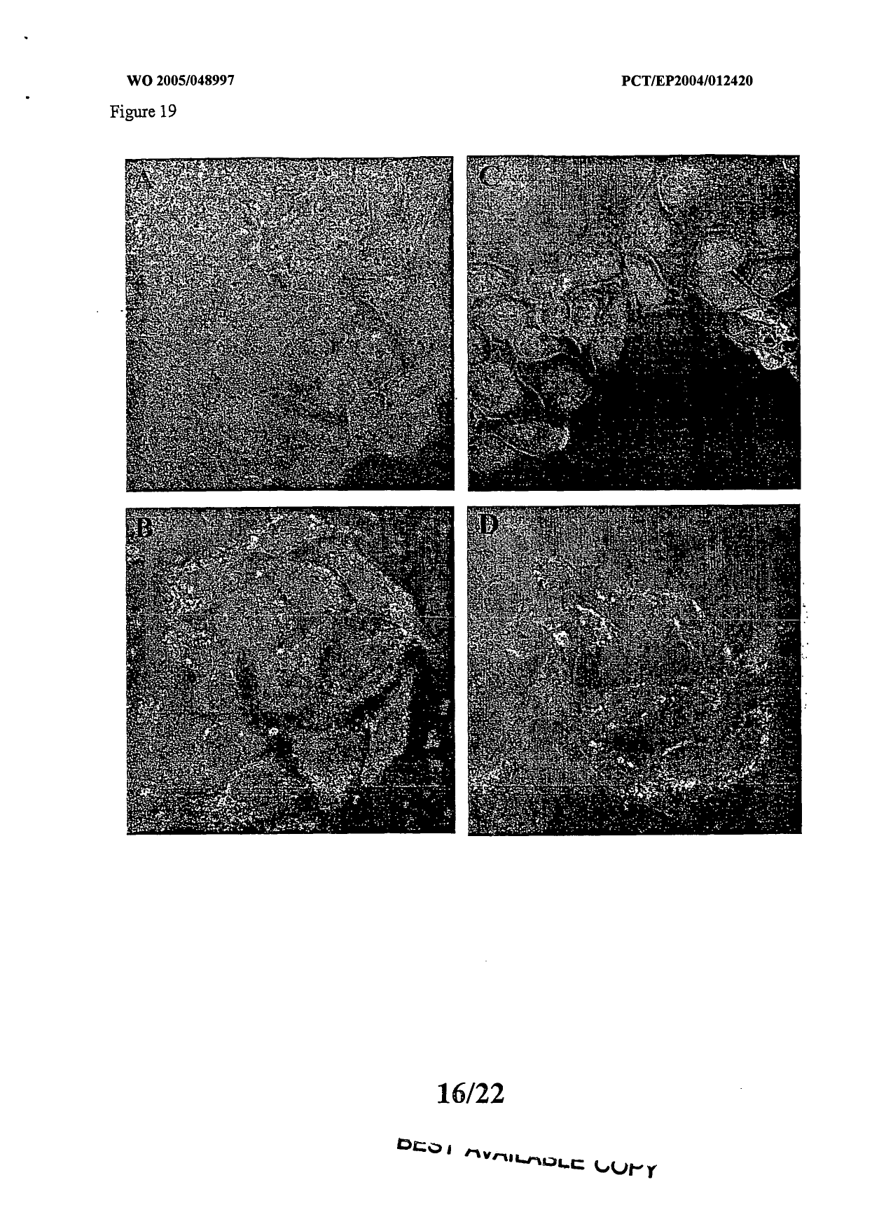

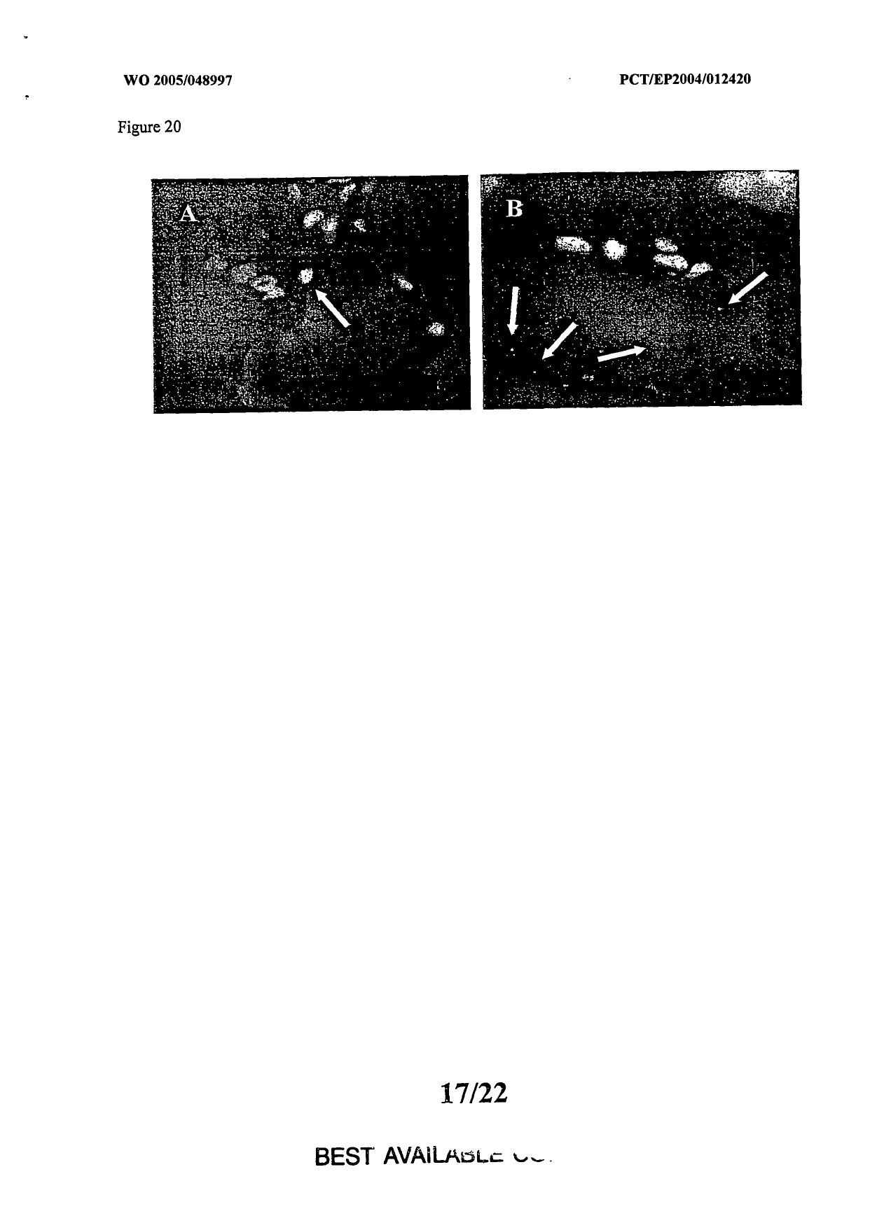

The capability of these nanoparticles to be internalized by the cells was evaluated using the PEG3-fluo sample. HL3T1 cells were incubated with PEG3-fluo alone or complexed with pCV-0 plasmid DNA, fixed with paraformaldehyde and analyzed after 2 and 24 hours incubation. Confocal microscopic analysis showed that after 2 hours incubation, a very low amount of both nanoparticles alone (FIG. 19A) and DNA/nanoparticle complexes (FIG. 19C) were detected in the cells. However, after 24 hours, the nanoparticles (FIG. 19B) and the DNA/nanoparticle complexes (FIG. 19D) were completely internalized by the cells, with similar tranfection efficiencies.

Finally, to determine whether the nanoparticles are taken up by the cells in vivo, mice were injected intramuscularly with the PEG3-fluo sample and sacrificed 15 minutes or 30 minutes after injection for analysis at a fluorescent microscope of the muscle at the site of suggesting that these nanoparticles may represent a useful delivery system for DNA vaccine application.

### Example 12

Evaluation of Gene Expression in Vitro

Uptake, release and expression of plasmid pGL2-CMV-Luc-basic from the DNA/nanoparticle complexes was evaluated in HeLa cells. Cells (5×10⁵) were seeded in 60-mm Petri dishes and cultured at 37° C. Twenty-four hours later, cells were incubated with the DNA/nanoparticle complexes, prepared as described in Example 9, and resuspended in 100 μl of DMEM containing 10% FBS. Controls were represented by cells incubated with naked DNA, or transfected with 1 μg of DNA using the calcium phosphate co-precipitation technique, and untreated cells. Forty-eight hours later the expression of the reporter genes was measured on amounts of cell extracts normalized to total protein contents, as previously described (Betti M, et al., supra). Expression of luciferase was evaluated using the Luciferase Assay Systems, (E1500, Promega), according to the manufacture's instructions, and read with a TD-20/20 Luminometer (TurnerDesigns, Sunnyvale, Calif.).

To assess stability of the DNA/nanoparticle formulations, in some experiments, pGL2-CMV-Luc-basic/PEG32 complexes were prepared, as described in Example 9, lyophilized, stored in a powder form at room temperature (25°-30° C.) for 1 month, and resuspended in the appropriate volume of 20 mM sodium phosphate buffer. After stirring for 1 hour, the complexes were added to the cells to evaluate gene expression as described above.

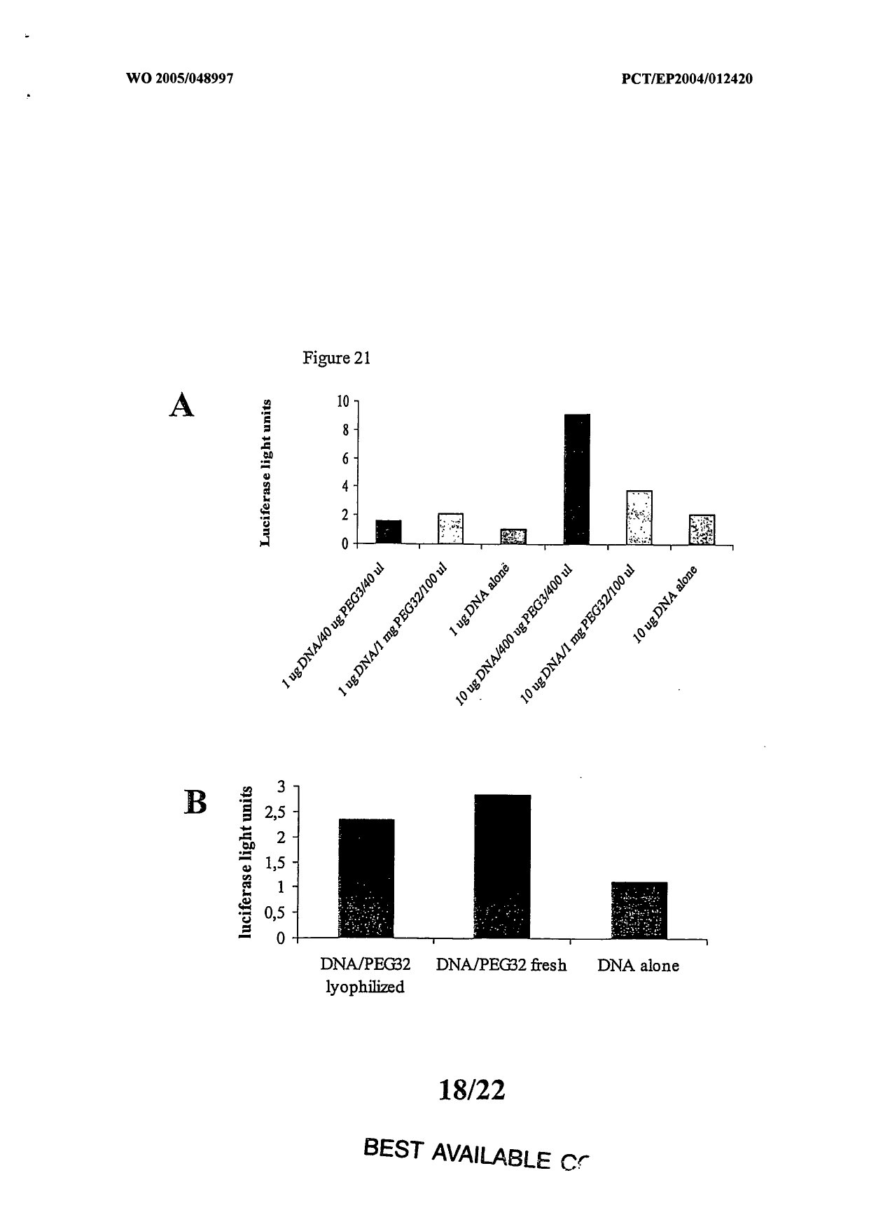

The capability of the DNA/nanoparticle complexes to release DNA and to allow its expression intracellularly was evaluated in HeLa cells incubated for 48 hours with pGL2-CMV-Luc plasmid DNA (1 or 10 μg) naked or associated to PEG3 (ratio 25 μg/mg/ml) or PEG32 (ratios of 10 or 100 μg/10 mg/ml) nanoparticles. As shown in FIG. 21A, luciferase gene expression was higher in cells incubated with the DNA/nanoparticle complexes as compared to cells incubated with naked DNA. These results indicate that the complexes are taken up by the cells and release functional DNA.

Finally, to determine whether the DNA/nanoparticle complexes are stable after storage at room temperature, pGL2CMV-Luc plasmid DNA/PEG32 nanoparticle formulations (ratio 100 μg/10 mg/ml) were lyophilized, stored at room temperature for 1 month, resuspended in 20 mM phosphate buffer and tested for gene expression. Controls were represented by cells treated with the same formulation freshly-prepared. The results, shown in FIG. 21B, indicate that phosphate buffer and tested for gene expression. Controls were represented by cells treated with the same formulation freshly-prepared. The results, shown in FIG. 21B, indicate that expression of the luciferase gene was similar in cells treated with the formulation freshly-prepared and immediately added to the cells as compared to cells treated with the same formulation lyophilized and stored at room temperature for 1 month. These results demonstrate that the DNA/nanoparticle complexes are stable in a powder form at room temperature.

### Reference Example 3

Tat Protein and Peptides

The 86-aa long at protein (HTLVIIIB, BH-10 clone) was expressed in Escherichia coli and isolated by successive rounds of high pressure chromatography and ion-exchange chromatography (see Chang H. C. et al., AIDS 11: 1421-1431, 1997; Chang H C et al., J. Biom. Sci 2: 189-202, 1995; Ensoli B. et al., Nature 345:84-86, 1990; Ensoli B. et al., J. Virol. 67: 277-87, 1993; Fanales-Belasio E. et al., J. Immunol. 168: 197-206, 202). The purified Tat protein is >95% pure as tested by SDS-PAGE, and HPLC analysis. To prevent oxidation that occurs easily because Tat contains seven cysteines, the Tat protein was stored lyophilized at −80° C. and resuspended in degassed sterile PBS (2 mg/ml) immediately before use. In addition, since Tat is photo- and thermo-sensitive, the handling of Tat was always performed in the dark and on ice. Peptides were synthesized by UFPeptides s.r.l. (Ferrara, Italy). Peptide stocks were prepared in DMSO at 10⁻² M concentration, kept at −80° C., and diluted in PBS immediately before use. Tat predicted CTL epitopes were selected using a peptide binding predictions program (http://bimas.dcrt.nih.gov/molbio/hla_bind).

### Example 13

Mice Immunization and Analysis of Immune Response

Animal use was according to national guidelines and institutional policies. Seven-weeks-old female BALB/c (H^{2kd}) mice (Harlan, Udine, Italy) were immunized with 100 μl of plasmid pCV-tat (1 μg), alone or complexed with the PEG32 nanoparticles (1 mg). The immunogens were given by bilateral intramuscular (i.m.) injections in the quadriceps muscles of the posterior legs (50 μl/leg). Control animals included mice injected with plasmid pCV-0 (1 μg) alone or associated to the nanoparticles. Animals were immunized with the DNA/nanoparticle complexes or with the DNA alone at weeks 0 and 4, and boosted with Tat protein (1 μg) in Alum, at weeks 8 and 16 after the first immunization. Mice were sacrificed 10 days after the last boost to collect blood and organs for analysis of humoral and cellular responses, and for histological, histochemical and immunoistochemical studies. During the course of the experiments, animals were controlled twice a week at the site of injection and for their general conditions (such as liveliness, food intake, vitality, weight, motility, sheen of hair). No signs of local nor systemic adverse reactions were ever observed in mice receiving the DNA/nanoparticle complexes as compared to mice vaccinated with naked DNA, or to untreated mice. Experiments were run in duplicate.

Serological response against Tat was measured by enzyme-linked immunosorbent assay (ELISA) using 96-well immunoplates (Nunc-immunoplate F96 PolySorb, Nalge Nunc International, Hereford, UK) coated with 100 μl/well of Tat protein (1 μg/ml in 0.05 M carbonate buffer pH 9.6-9.8) for 16 hours at 4° C. (see reference example 3). Wells were washed with 0.05% Tween 20 in PBS (PBS-Tween) in an automated washer (Immunowash 1575, BioRad Laboratories) and blocked with PBS containing 3% BSA (Sigma, St. Louise, Mich.) for 90 minutes at 37° C. Sera were diluted in PBS containing 3% BSA. The lowest serum dilution was 1:100 (duplicate wells). After extensive washing, 100 μl aliquots were added to each well in duplicate and incubated for 90 minutes at 37° C. Plates were washed and 100 μl/well of horse-radish peroxidase-conjugated sheep anti-mouse IgG (Amersham Pharmacia Biotech, Uppsala, Sweden), diluted 1:1000 in PBS-Tween containing 1% BSA, were added. After incubation for 90 minutes at room temperature, plates were washed and incubated with peroxidase substrate (ABTS) (Roche) for 40 minutes at room temperature. The reaction was blocked with 0.1 M citric acid and the absorbency was measured at 405 nm in an automated plate reader (ELX-800, Bio-Tek Instruments, Winooski, Utah). The cutoff corresponded to the mean OD₄₀₅ (+3 SD) of sera of control mice, tested in three independent assays.

For anti-Tat IgG epitope mapping, eight synthetic peptides (aa 1-20, 21-40, 36-50, 46-60, 56-70, 52-72, 65-80, 73-86) representing different regions of Tat (HTLVIII-BH10) were diluted in 0.1 M carbonate buffer (pH 9.6) at 10 μg/ml, and 96-well immunoplates were coated with 100 μl/well. The assays were performed as described above. The cutoff for each peptide corresponded to the mean OD₄₀₅ (+3 SD) of sera of control mice injected with PBS, tested in three independent assays.

For anti-Tat IgG isotyping, plates were coated with Tat protein and incubated with mice sera diluted 1:100 and 1:200, as described above. After washing, 100 μl of goat anti-mouse IgG1, or IgG2a (Sigma), diluted 1:100 in PBS-Tween containing 1% BSA, were added to each well. Immunocomplexes were detected with a horse-radish peroxidase-labeled rabbit anti-goat IgG (Sigma) diluted 1:7500 in PBS-Tween containing 1% BSA, as described above. The cutoff for each IgG subclass corresponded to the mean OD₄₀₅ (+3 SD) of sera of control mice injected with PBS, tested in three independent assays.

The presence of anti-Tat specific antibodies was evaluated by ELISA assays. Anti-Tat IgG were detected after immunization with pCV-tat DNA/PEG32 complexes (mean titers 2738±2591), in a fashion similar to mice immunized with the same prime/boost regimen but with naked DNA (mean titers 4686±5261) (p<0.05) (Table 9).

TABLE 9
Analysis of anti-Tat humoral response^{a}

| | IgG Titer | | IgG Isotype |
|---|---|---|---|
| | (Number of | | (IgG1/IgG2a) |
| | responding mice/group) | | IV° |
| Group | III° immunization | IV° immunization | immunization |
| pCV-tat/PEG32 | 780 | 2733.8 ± 2591.7 | 1.30 |
| (1 μg/mg) | (1/4) | (4/4) | |
| pCV-tat | 1560 | 4686.3 ± 5261.1 | 1.25 |
| (1 μg) | (1/4) | (4/4) | |
| ^{a}Mice were immunized i.m. with pCV-tat DNA/PEG32 complexes or with pCV-tat DNA alone at weeks 0 and 4, and boosted with Tat protein (1 μg) in Alum at weeks 8 and 16. Anti-Tat IgG titers were tested after the first (III° immunization) and the second protein boost (IV° immunization) on single mice sera. The results correspond to mean titers (±SD) of mice sera per experimental group. Analysis of the IgG isotypes was performed after the second protein | | | |
| # boost (IV° immunization). The results represent the ratio between the mean OD_{405 nm} values of mice IgG1/IgG2 per experimental group. | | | |

The IgG isotype analysis indicated the presence of both IgG1 and IgG2a subclasses, with similar IgG1/IgG2a ratios and a slightly higher prevalence of IgG1 in both groups of mice immunized with naked DNA or with DNA/nanoparticle complexes (Table 9). The epitope reactivity of the antibodies was mainly directed against the amino-terminal (aa 1-20) and the carboxy-terminal (aa 65-80) regions of Tat (Table 10).

TABLE 10
Epitope mapping analysis of anti-Tat IgG^{a}

| | Peptide (aa) | | | | | | | |
|---|---|---|---|---|---|---|---|---|
| Group | 1-20 | 21-40 | 52-72 | 73-86 | 36-50 | 46-60 | 56-70 | 65-80 |
| pCV- | 0.217 ± 0.16 | 0.011 ± 0 | 0.001 ± 0 | 0.009 ± 0 | 0.008 ± 0 | 0.008 ± 0 | 0.009 ± 0 | 0.111 ± 0.10 |
| tat/PEG32 | | | | | | | | |
| (1 μg/mg) | | | | | | | | |
| PCV-tat | 0.301 ± 0.23 | 0.003 ± 0 | 0.007 ± 0 | 0.017 ± 0 | 0.079 ± 0.1 | 0.022 ± 0 | 0.007 ± 0 | 0.110 ± 0.05 |
| (1 μg) | | | | | | | | |

^{a}Mice were immunized i.m. with pCV-tat DNA/PEG32 complexes or with pCV-tat DNA alone at weeks 0 and 4, and boosted with Tat protein (1 μg) in Alum at weeks 8 and 16. Analysis of the IgG epitope reactivity was performed after the second protein boost (IV° immunization). The results correspond to the mean OD_{405 nm} values (± SD) of mice sera per experimental group. The cutoff values for each peptide were ≦0.02.

Tat-Specific T Cell Proliferation

Splenocytes were purified from spleens squeezed on filters (Cell Strainer, 70 μM, Nylon, Becton Dickinson). Following red blood cell lysis with of 154 mM NH₄Cl, 10 mM KHCO₃ and 0.1 mM EDTA (5 ml/spleen) for 4 minutes at room temperature, cells were diluted with RPMI 1640 containing 3% FBS (Hyclone), spun for 10 minutes at 1200 rpm, resuspended in RPMI 1640 containing 10% FBS and used for the analysis of antigen-specific cellular immune responses. Pool of spleens per each experimental group were used.

Splenocytes (2.5×10⁵/100 μl) were cultured in 96-well plates in the presence of affinity-purified Tat protein (0.1, 1, or 5 μg/ml) or Concanavaline A (2 μg/ml, Sigma) for 4 days at 37° C. [methyl-³H]-Thymidine (2.0 Ci/mmol, NEN-DuPont, Boston, Mass.) was added to each well (0.5 μCi), and cells were incubated for 16 hours at 37° C. [³H]-Thymidine incorporation was measured with a β-counter (Top Count, Packard). The stimulation index (SI) was calculated by dividing the mean counts/min of six wells of antigen-stimulated cells by the mean counts/min of the same cells grown in the absence of the antigen.

CD4+ T-cell proliferation in response to Tat was evaluated using mice splenocytes, cultured for five days in the presence of 0.1, 1 and 5 μg/ml of Tat protein. Antigen-stimulated T-cell proliferation, determined by [³H]thymidine incorporation, was similarly detected in both groups of mice immunized with pCV-tat/PEG32 and pCV-tat alone (Table 11).

TABLE 11
Lymphoproliferative response to Tat^{a}

| | Tat (μg/ml) | | | ConA (μg/ml) |
|---|---|---|---|---|
| Group | 0.1 | 1 | 5 | 2 |
| pCV-tat/PEG32 | 1.35 | 2.02 | 2.72 | 12.60 |
| (1 μg/mg) | | | | |
| pCV-tat | 1.46 | 1.86 | 3 | 8.82 |
| (1 μg) | | | | |

^{a}Values represent the SI of murine splenocytes after Tat protein (0.1, 1 or 5 μg/ml) or ConA addition.

CTL Assays

CTL assays were carried out on B-depleted splenocytes. Depletion of B lymphocytes was carried out using anti-CD19 magnetic beads (Becton Dickinson, Milan, Italy), according to the manufacturer's instructions. Fluorescence-activated cell sorter (FACS) analysis was carried out on cells (1×10⁶) washed with PBS, without calcium and magnesium, containing 1% BSA (washing buffer). The cellular pellet was pre-incubated with 10 μl of a mouse pre-immune serum to saturate unspecific binding, for 2 min at 4° C., and then incubated with 1 μg of rat anti-mouse monoclonal antibodies (α-CD19, α-CD3, α-CD4, α-CD8) (Becton Dickinson) for 45 min at 4° C. After extensive washing, cells were incubated with 1 μg of a goat anti-rat FITC-conjugated antibody (Becton Dickinson) for 30 min, washed and resuspended in 400 μL of washing buffer. In some experiments, cells were stained with a fluorocrome-conjugated primary antibody (α-CD19-PE, α-CD3-FITC, α-CD8-PE, Becton Dickinson). Sample fluorescence was measured using a FACSCalibur from Becton Dickinson. Splenocytes were co-cultivated with Balb/c 3T3 Tat cells (ratio 5:1), previously irradiated with 30 Gy (¹³⁷Cs). After 3 days, rIL-2 (10 U/ml) (Roche) was added and cells co-cultivated for additional 3 days at 37° C. Dead cells were then removed by Ficol gradient (Histopaque, Sigma). CTL activity was determined, at various effector/target ratios, by standard ⁵¹Cr release assays using syngeneic P815 target cells, previously labeled with ⁵¹Cr (25 μCi/3×10⁶ cells; NEN-DuPont) for 90 minutes at 37° C., and pulsed with Tat peptides (1×10⁻⁵ M), containing Tat computer predicted CTL epitopes, for 1 hour at 37° C. After 5 hours incubation at 37° C., the percentage of ⁵¹Cr release was determined in the medium. Percent (%) of specific lysis was calculated as 100×(cpm sample−cpm medium)/(cpm Triton-X100−cpm medium). Spontaneous release was below 10%.

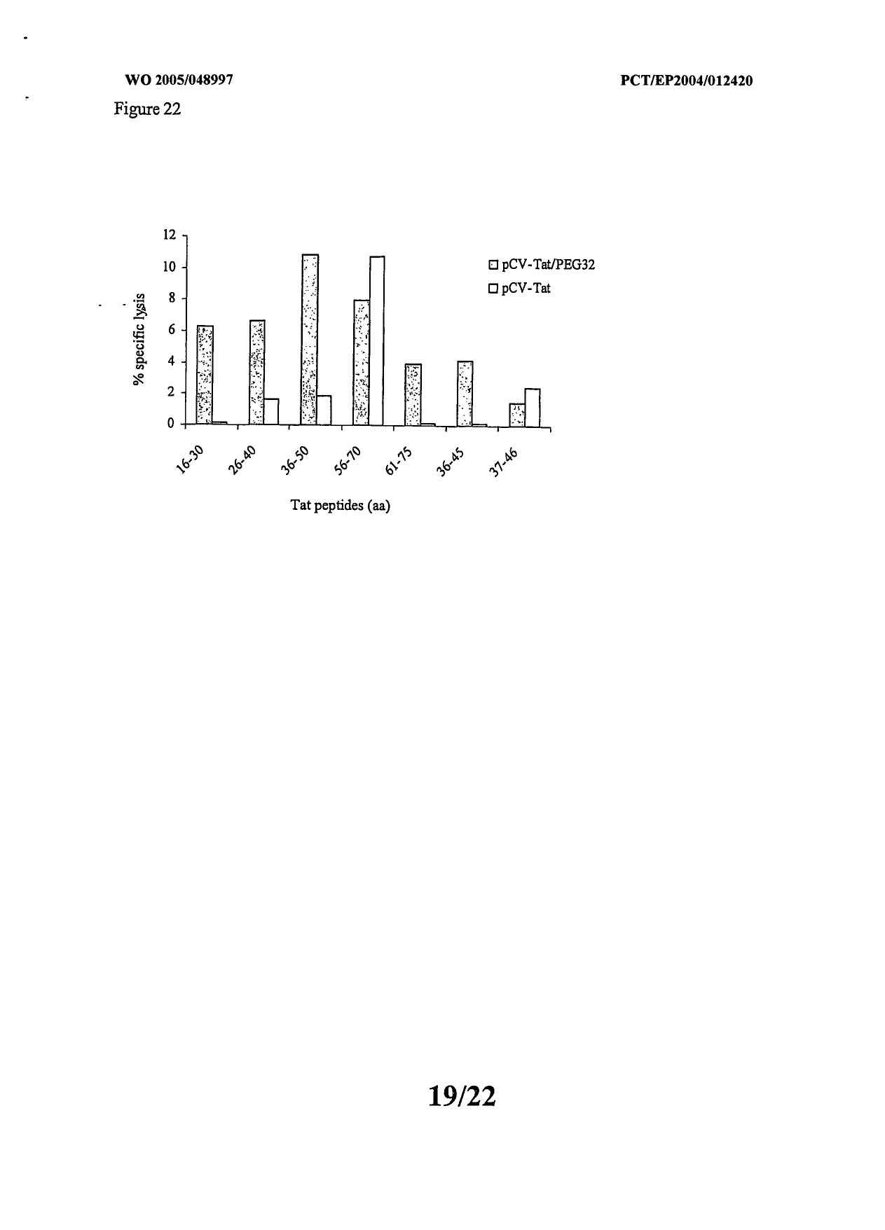

As shown in FIG. 22, a specific anti-Tat CTL activity, directed against P815 target cells pulsed with Tat peptides containing computer predicted CTL epitopes, was detected in both groups of mice immunized with tat/PEG32 and with tat DNA alone. However, the CTL response was generally higher (in terms of percent of specific lysis) and broader (in terms of target epitopes) in mice vaccinated with the tat/PEG32 complexes as compared to mice vaccinated with naked DNA.

Histological, Histochemical and Immunohistochemical Procedures

At sacrifice animals were subjected to autopsy. Sample of cutis, subcutis and skeletal muscles at the site of injection and other organs (lungs, heart, intestine, kidneys, spleen and liver) were taken and processed for histologic, histochemical and immunohistochemical examination, after fixation in 10% formalin for 12-24 h and embedding in paraffin. Three-5 μm paraffin-embedded sections were stained with hematoxylin and eosin, subjected to periodic acid Shiff (PAS) reaction without or with diastase (Sigma) treatment, and to Pearl's reaction for ferric iron. The avidin-biotin-peroxidase complex technique was used for the immunohistochemical studies performed on paraffin sections. The panel of antibodies included S-100 (DAKO, Glostrup, Denmark), HH-F 35 (DAKO) for detection of α-actin, CD68 and Mac387 (DAKO) for detection of macrophages. Briefly, after deparaffinization and rehydration, endogenous peroxidase was blocked with 0.3% H₂O₂ in methanol; samples were then incubated with primary antibodies for 10-12 h at 4° C. Biotinilated-anti-mouse and anti-rabbit immunoglobulins (Sigma) were utilized as secondary antibodies. Specific reactions were detected following incubation with avidin-biotin-peroxidase conjugated and development in diaminobenzidine (Sigma).

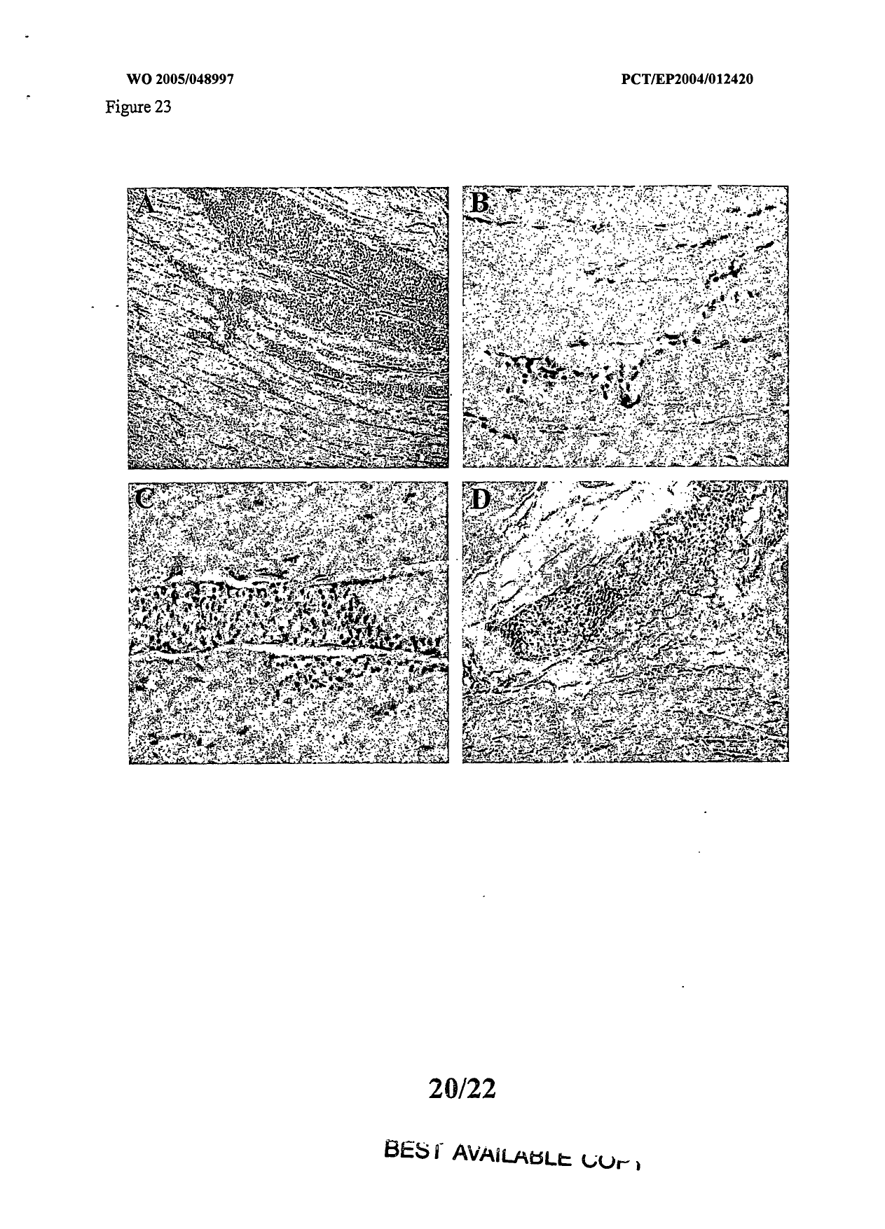

To assess safety of these novel core-shell nanoparticles, mice were controlled after immunization twice a week at the site of injection and for their general health conditions. No signs of local nor systemic adverse reactions were ever observed in mice receiving the tat/PEG32 complexes, as compared to control mice injected with naked DNA or untreated mice. An inflammatory reaction at the site of injection, mainly characterized by macrophage infiltration, was observed in 87.5% and 85.7% of mice treated with the pCV-tat/PEG32 and pCV-0/PEG32 complexes, respectively, and in 75% and 80% of mice immunized with naked pCV-tat and pCV-0 DNA, respectively, indicating that that there are no differences in the frequency of inflammatory reactions (at the intramuscular level) in mice injected with the complexes as compared to animals inoculated with naked DNA. As shown in FIG. 23, macrophages infiltration, with variable intensity, was observed in the fibroadipose tissue around the muscle fibers at the site of injection and among the muscle fibers (FIG. 23A). The muscular inflammatory infiltration sometimes was light and without regressive alterations of muscle fibers (FIG. 23B), sometimes it was associated to regressive changes (FIG. 23C). Sometimes macrophages were observed in the adipose tissue surrounding the injection site (FIG. 23D). Finally, no specific alterations that may be related to injection of DNA/nanoparticle complexes were reported in the other organs examined, as compared to mice injected with naked DNA.

In this statistical analysis of the results the students τ test was performed in accordance with the principles and practice of the statistics in biological research.

The present inventors have designed and synthesized by emulsion polymerization novel anionic core-shell nanoparticles such as those described in Examples 9 to 13 for the delivery of DNA. These nanoparticles have an inner hard core costituted of poly(methyl methacrylate) and highly hydrophilic outer shell constituted by hydrosoluble copolymers bearing positively charged functional groups, able to reversibly bind DNA, and by polyethylenglycol chain brushes, able to increase their biocompatibility. As a result of the polymerisation mechanism, in the core-shell nanoparticles described in this study the charged macromolecules are not simply adsorbed, but covalently bound to the particle surface, thus avoiding physical desorption and/or instability/toxicity drawbacks associated with vaccine formulations containing free surfactants and/or detergents.

The results of Examples 9 to 13 indicate that the DNA is adsorbed with high efficiency (80%-100% of DNA initially incubated with the nanoparticles) onto the nanoparticles surface. The DNA release in vitro occurs already after 10 minutes of complex incubation at 37° C. and it is time-dependent and long-lasting. Finally, the DNA preserves its structural integrity.

The studies in tissue culture systems showed that they are taken up by the cells, and that cellular internalization of the DNA/nanoparticle complexes is similar to that of the nanoparticles alone, indicating that the presence of DNA onto the nanoparticle surface does not interfere with cellular internalization. In addition, the studies in cell cultures demonstrated that these nanoparticle/DNA complexes release functional DNA. Finally, it was shown that the DNA/nanoparticle formulations are stable in a powder-form at room temperature for at least 1 month. Indeed, after suspension of the powder in physiological buffer, the DNA/nanoparticle complexes retained their capacity to be taken up by the cells and to release functional DNA, in a fashion similar to freshly prepared DNA-nanoparticle formulations. The safety studies showed that they are not toxic in vitro nor in mice, even after multiple administration of high doses (1 mg). Finally, the immunogenicity studies showed that vaccination with a low dose (1 μg) of plasmid DNA and a prime-boost regimen elicits broad humoral and cellular responses against the antigen of both Th1 and Th2 type. Of note, immunization with the DNA/nanoparticle complexes elicits broader CTL responses in terms of percentage of specific lysis and of epitope reactivity.

In conclusion, the results indicate that these innovative nanoparticles represent a promising tool for the development of novel and stable DNA vaccines characterized by low cost, increased shelf-life, safety, suitability for scale-up and GMP production, ease of administration and feasibility for technology transfer to developing countries.

### Example 14

### ZP3 Nanoparticles

Analysis of Cytotoxicity in Vitro

Monolayer cultures of HL3T1 cells were obtained through the American Type Cell culture collection (ATCC) and grown in DMEM (Gibco, Grand Island, N.Y.) containing 10% FBS (Hyclone, Logan, Utah) [Wright C M Science 234:988-92, 1986]. Cells (1×10^{4/}100 μl) were seeded in 96-well plates and cultured at 37° C. for 24 hours. Medium was then replaced with 100 μl of medium containing increasing concentrations of ZP3 nanoparticles (500-10.000 μg/ml). Each sample was assayed in sextupled wells. Cells were incubated for 96 hours at 37° C., and cell proliferation was measured using the colorimetric cell proliferation kit I (MTT based) (Roche, Milan, Italy) [Mossman T. et al, supra], according to manufacturer's instructions, and compared to that of untreated cells. Statistical analysis (τ-student) was performed.

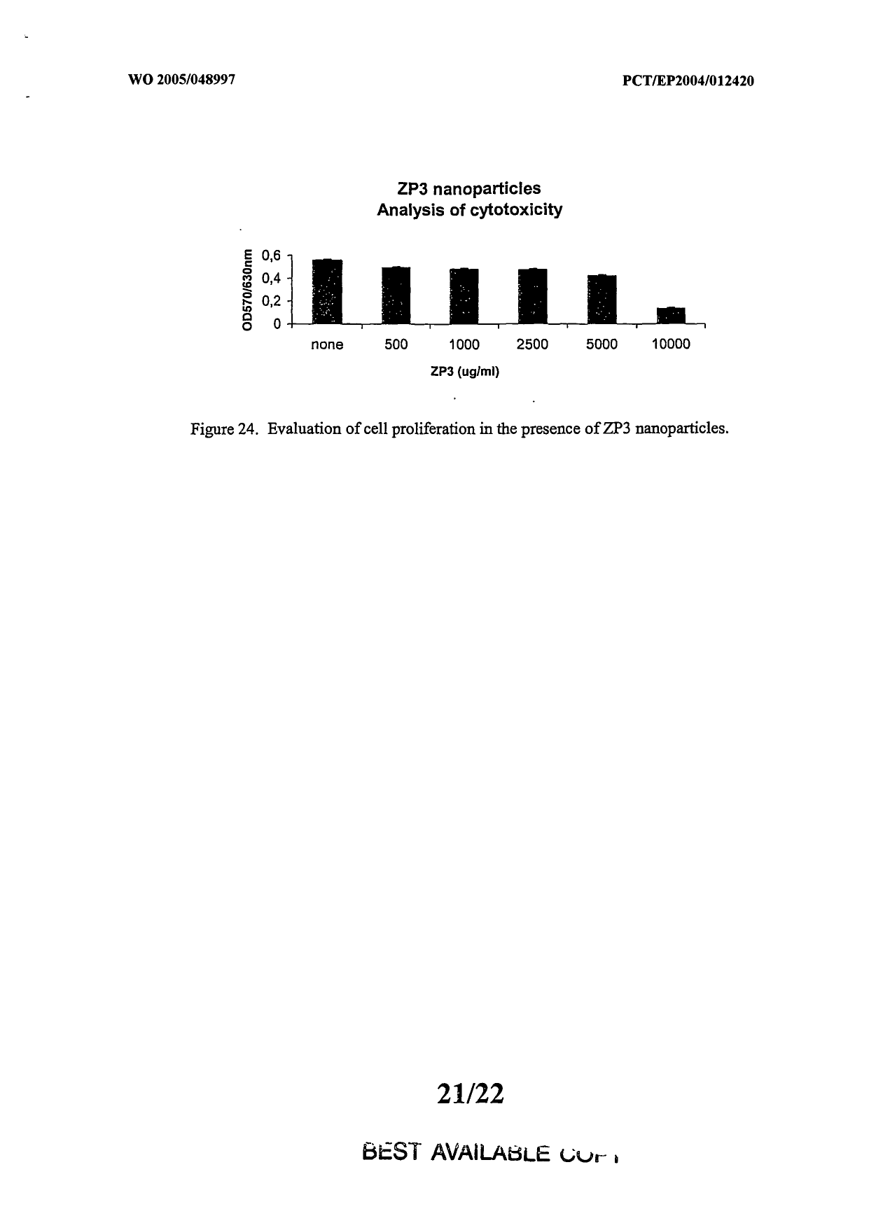

As shown in FIG. 24, no significant reduction (p>0.05) of cell viability was observed up to 2500 μg/ml after 96 hours incubation in the samples treated both with ZP3, as compared to untreated cells. A 25% and 76% reduction in cell viability was observed in the presence of 5 and 10 mg/ml, respectively, of ZP3. These results indicate that ZP3 nanoparticles are not toxic for the cells even at very high doses (in the milligram range).

### Example 15

### MA7 Nanoparticles

Analysis of Cytotoxicity in Vitro

Monolayer cultures of HL3T1 cells, containing an integrated copy of plasmid HIV-1-LTR-CAT, where expression of the chloramphenicol acetyl transferase (CAT) reporter gene is driven by the HIV-1 LTR promoter, were obtained through the American Type Cell culture collection (ATCC) and grown in DMEM (Gibco, Grand Island, N.Y.) containing 10% FBS (Hyclone, Logan, Utah). Cells (4×10³/100 μl) were seeded in 96-well plates and cultured at 37° C. for 24 h. One hundred μl of medium containing the nanoparticles alone (10-500 μg/ml) or bound to Tat (1 μg/ml) were added to the cells in sextuplicate wells. Untreated cells and cells grown with Tat alone were used as controls. After 96 h incubation at 37° C., cell viability was measured using the colorimetric cell proliferation kit I (MTT-based) provided by Roche (Milan, Italy). Absorbances were measured by reading the plates at 570 nm with reference wavelength at 630 nm (OD 570/630). τ-student tests were performed.

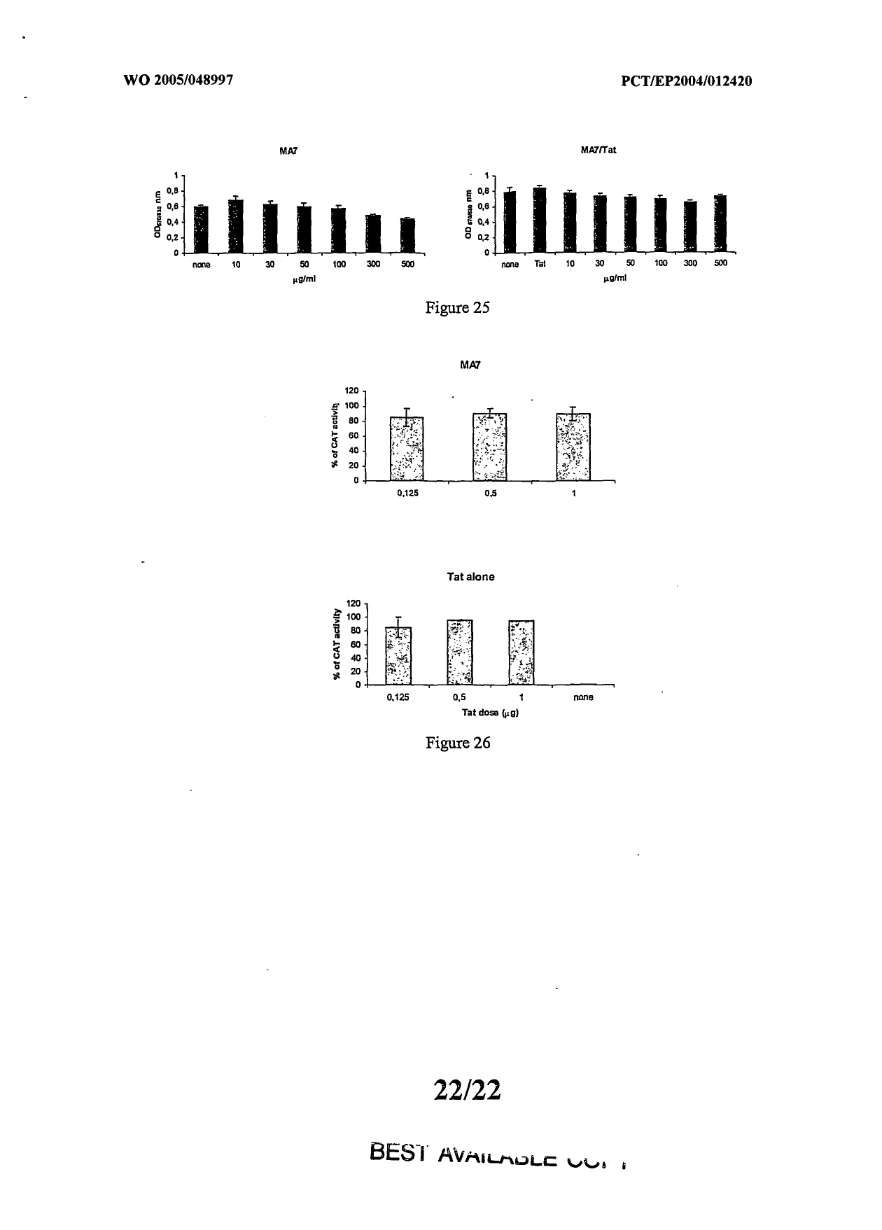

Thus, the cytotoxicity of MA7 was assayed in HL3T1 cells following incubation with increasing amounts of nanoparticles (10-500 μg/ml) as compared to untreated cells. As shown in FIG. 25, no significant reduction of cell viability was observed after 96 hours incubation in the samples treated with MA7, as compared to untreated cells. These results indicate that MA7 nanoparticles are not toxic for the cells.

Nanoparticle/Tat Protein Complex Formation and Evaluation of Tat Protein Activity

The 86-aa long Tat protein (HTLVIII, BH-10 clone) was expressed in Escherichia coil and isolated by successive rounds of high pressure chromatography and ion-exchange chromatography, as described in Reference Example 3. The purified Tat protein is >95% pure as tested by SDS-PAGE, and HPLC analysis. To prevent oxidation that occurs easily because Tat contains seven cysteines, the Tat protein was stored lyophilized at −80° C. and resuspended in degassed sterile PBS (2 mg/ml) immediately before use. In addition, since Tat is photo- and thermo-sensitive, the handling of Tat was always performed in the dark and on ice.

Nanoparticles (lyophilized powder) were resuspended in sterile PBS at 2 mg/ml at least 24 hours before use. The appropriate volumes of Tat and nanoparticles were mixed and incubated in the dark and on ice for 60 minutes. After incubation samples were spun at 15.500 rpm for 10 minutes. The pellets (Tat-nanoparticle complexes) were resuspended in the appropriate volume of degassed sterile PBS and used immediately.

HL3T1 cells (5×10⁵) were seeded in 6-well plates. Twenty-four h later cells were replaced with 1 ml of fresh medium and incubated with Tat alone (0.125, 0.5 and 1 μg/ml) or with Tat bound to the nanoparticles (30 μg/ml) in the presence of 100 μM chloroquine (Sigma, St. Louise, Mich.). CAT activity was measured 48 h later in cell extracts after normalization to total protein content, as described previously.

For their application as delivery systems in vaccine development, polymeric microspheres should bind and release a protein in its biologically active conformation. This is particularly important for Tat since a native protein is required for vaccine efficacy. Therefore, the capability of the nanoparticles to bind and release the HIV-1 Tat protein in its biologically active conformation was determined in HL3T1 cells, containing an integrated copy of the reporter plasmid HIV-1 LTR-CAT. In these cells expression of the CAT gene occurs only in the presence of bioactive Tat. To this purpose, HL3T1 cells were incubated with increasing amounts of Tat alone or Tat adsorbed onto MA7. The results are depicted in FIG. 26. Expression of CAT was high and similar between samples incubated with MA7/Tat and Tat alone. These results demonstrate that the nanoparticles adsorb and release biologically active Tat protein, and that Tat bound to the microspheres maintains its native conformation and biological activity.

1. Core-shell nanoparticles comprising:
(a) a core which comprises a water insoluble polymer or copolymer, and
(b) a shell which comprises a hydrophilic polymer or copolymer;
said nanoparticles being obtainable by emulsion polymerization of a mixture comprising, in an aqueous solution, at least one water-insoluble styrenic, acrylic or methacrylic monomer and:
(i) a monomer of formula (I): 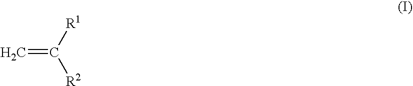
wherein
R¹ represents hydrogen or methyl, and
R² represents —COOAOH, —COO—A—NR⁹R¹⁰ or —COO—A—N⁺R⁹R¹⁰R¹¹X⁻, in which A represents C₁₋₂₀ alkylene, R⁹, R¹⁰ and R¹¹ each independently represent hydrogen or C₁₋₂₀ alkyl and X represents halogen, sulphate, sulphonate or perchlorate, and
a water-soluble polymer of formula (II) 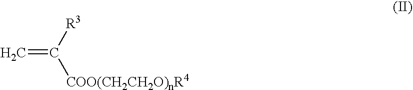
wherein
R³ represents hydrogen or methyl,
R⁴ represents hydrogen or C₁₋₂₀ alkyl, and
n is an integer such that the polymer of formula (I) has a number-average molecular weight of at least 1000; or
(ii) a hydrophilic copolymer which comprises repeating units of formulae (III) and (IV): 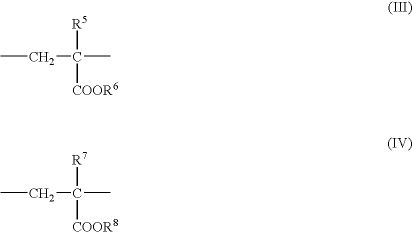
wherein
R⁵ and R⁷ each independently represent hydrogen or methyl,
R⁶ represents hydrogen, —A—NR⁹R¹⁰ or —A—N⁺R⁹R¹⁰R¹¹X⁻, in which A represents C₁₋₂₀ alkylene, R⁹, R¹⁰ and R¹¹ each independently represent hydrogen or C₁₋₂₀ alkyl and X represents halogen, sulphate, sulphonate or perchlorate and
R⁸ represents C₁₋₁₀ alkyl.

2. Nanoparticles according to claim 1 wherein the core comprises poly(C₁₋₁₀ alkyl (meth)acrylate), polystyrene or a copolymer formed from monomers which are acrylic, methacrylic or styrenic monomers.

3. Nanoparticles according to claim 1 wherein the core comprises poly(methyl methacrylate).

4. Nanoparticles according to claim 1 which are obtainable by emulsion polymerization of methyl methacrylate in an aqueous solution comprising poly(ethylene glycol) methyl ether methacrylate and 2-(dimethyloctyl) ammonium ethyl methacrylate bromine.

5. Nanoparticles according to claim 1 which are obtainable by emulsion polymerization of methyl methacrylate in an aqueous solution comprising a copolymer of methacrylic acid and ethyl acrylate.

6. Nanoparticles according to claim 1 to which are obtainable by emulsion polymerization of methyl methacrylate in an aqueous solution comprising a copolymer of 2-(dimethylamino)ethyl methacrylate and C₁₋₆ alkyl methacrylate.

7. Nanoparticles according to claim 1 which have a number-average particle diameter measured by scanning electron microscopy of from 50 to 1000 nm.

8. Nanoparticles according to claim 1 which further comprise a fluorescent chromophore.

9. A process for preparing nanoparticles according to claim 1, said process comprising emulsion polymerization of a water-insoluble monomer in an aqueous solution comprising:
(i) a monomer of formula (I) and a polymer of formula (II), or
(ii) a hydrophilic copolymer which comprises repeating units of formulae (III) and (IV).

10. Nanoparticles according to claim 1 which further comprise at least one pharmacologically active agent adsorbed at the surface of the nanoparticles.

11. Nanoparticles according to claim 10 wherein the pharmacologically active agent is a disease-associated antigen.

12. Nanoparticles according to claim 11 wherein the antigen is a deoxyribonucleic acid, ribonucleic acid, oligodeoxynucleotide, oligonucleotide or protein.

13. Nanoparticles according to claim 11 wherein the antigen is a microbial antigen or a cancer-associated antigen.

14. Nanoparticles according to claim 11 wherein the antigen is a human immunodeficiency virus-1 (HIV-1) antigen.

15. Nanoparticles according to claim 14 wherein the antigen is HIV-1 Tat protein or an immunogenic fragment thereof.

16. A process for preparing nanoparticles which comprise at least one pharmacologically active agent adsorbed at the surface of the nanoparticles, said process comprising adsorbing a pharmacologically active agent at the surface of nanoparticles according to claim 1.

17. A pharmaceutical composition comprising nanoparticles according to claim 10 and a pharmaceutically acceptable excipient.

18. A method of diagnosing, treating or preventing a condition in a subject said method comprising administering an effective amount of nanoparticles according to claim 10 to a subject in need of such treatment.

19. A method according to claim 18, wherein the pharmacologically active agent is a disease-associated antigen and the nanoparticles are administered to the subject to generate an immune response in the subject.

20. A method according to claim 18, wherein the antigen is a human immunodeficiency virus-1 (HIV-1) antigen and the nanoparticles are administered to the subject to prevent or treat HIV infection or AIDS.

21-23. (canceled)


SEQUENCE LISTING (40 sequences)

SEQ ID NO: 1 - DNA, 309 bp, Human immunodeficiency virus
  CDS (1)..(309)
atggagccagtagatcctcgtctagagccctggaagcatccaggaagtcagcctaaaactgcttgtaccaattgctattgtaaaaagtgttgctttcattgccaagtttgtttcataacaaaagccttaggcatctcctacggcaggaagaagcggagacagcgtcgaagacctcctcaaggcagtcagactcatcaagtttctctatcaaagcaacccacctcccaatcccgaggggacccgacaggcccgaaggaacagaagaagaaggtggagagagagacagagacagatccggtccatcagtga

SEQ ID NO: 2 - protein, 102 aa, Human immunodeficiency virus
MEPVDPRLEPWKHPGSQPKTACTNCYCKKCCFHCQVCFITKALGISYGRKKRRQRRRPPQGSQTHQVSLSKQPTSQSRGDPTGPKEQKKKVERETETDPVHQ

SEQ ID NO: 3 - DNA, 261 bp, Human immunodeficiency virus
  CDS (1)..(261)
atggagccagtagatcctcgtctagagccctggaagcatccaggaagtcagcctaaaactgcttgtaccaattgctattgtaaaaagtgttgctttcattgccaagtttgtttcataacaaaagccttaggcatctcctacggcaggaagaagcggagacagcgtcgaagacctcctcaaggcagtcagactcatcaagtttctctatcaaagcaacccacctcccaatcccgaggggacccgacaggcccgaaggaatag

SEQ ID NO: 4 - protein, 86 aa, Human immunodeficiency virus
MEPVDPRLEPWKHPGSQPKTACTNCYCKKCCFHCQVCFITKALGISYGRKKRRQRRRPPQGSQTHQVSLSKQPTSQSRGDPTGPKE

SEQ ID NO: 5 - DNA, 261 bp, Human immunodeficiency virus
  CDS (1)..(261)
atggagccagtagatcctagactagagccctggaagcatccaggaagtcagcctaaaactgctggtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcagcccacctcccaatcccgaggggacccgacaggcccgaaggaatag

SEQ ID NO: 6 - protein, 86 aa, Human immunodeficiency virus
MEPVDPRLEPWKHPGSQPKTAGTNCYCKKCCFHCQVCFITKALGISYGRKKRRQRRRPPQGSQTHQVSLSKQPTSQSRGDPTGPKE

SEQ ID NO: 7 - DNA, 261 bp, Human immunodeficiency virus
  CDS (1)..(261)
atggagccagtagatcctagactagagccctggaagcatccaggaagtcagcctaaaactgcttgtaccaattgctattgtaaaaagtgttgctttcattgccaagtttgtttcataacagctgccttaggcatctcctatggcaggaagaagcggagacagcgacgaagacctcctcaaggcagtcagactcatcaagtttctctatcaaagcagcccacctcccaatcccgaggggacccgacaggcccgaaggaatag

SEQ ID NO: 8 - protein, 86 aa, Human immunodeficiency virus
MEPVDPRLEPWKHPGSQPKTACTNCYCKKCCFHCQVCFITAALGISYGRKKRRQRRRPPQGSQTHQVSLSKQPTSQSRGDPTGPKE

SEQ ID NO: 9 - DNA, 252 bp, Human immunodeficiency virus
  CDS (1)..(252)
atggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcagcccacctcccaatccccgacaggcccgaaggaatag

SEQ ID NO: 10 - protein, 83 aa, Human immunodeficiency virus
MEPVDPRLEPWKHPGSQPKTACTNCYCKKCCFHCQVCFITKALGISYGRKKRRQRRRPPQGSQTHQVSLSKQPTSQSPTGPKE

SEQ ID NO: 11 - DNA, 252 bp, Human immunodeficiency virus
  CDS (1)..(252)
atggagccagtagatcctagactagagccctggaagcatccaggaagtcagcctaaaactgcttgtaccaattgctattgtaaaaagtgttgctttcattgccaagtttgtttcataacagctgccttaggcatctcctatggcaggaagaagcggagacagcgacgaagacctcctcaaggcagtcagactcatcaagtttctctatcaaagcagcccacctcccaatccccgacaggcccgaaggaatag

SEQ ID NO: 12 - protein, 83 aa, Human immunodeficiency virus
MEPVDPRLEPWKHPGSQPKTACTNCYCKKCCFHCQVCFITAALGISYGRKKRRQRRRPPQGSQTHQVSLSKQPTSQSPTGPKE

SEQ ID NO: 13 - DNA, 306 bp, Human immunodeficiency virus
  CDS (1)..(306)
atggatccagtagatcctaacctagagccctggaaccatccgggaagtcagcctacaactgcttgtaacaagtgttactgtaaaaagtgttgctatcattgccaagtttgctttctgaacaaaggcttaggcatctcctatggcaggaagaagcggagacagcgacgaggaactcctcagagcagtaaggatcatcaaaatcctataccaaagcaacccataccccaaacccaaggggtctcgacaggcccggaagaatcgaagaagaaggtggagagcaaggcagagacagatcgattcgattag

SEQ ID NO: 14 - protein, 101 aa, Human immunodeficiency virus
MDPVDPNLEPWNHPGSQPTTACNKCYCKKCCYHCQVCFLNKGLGISYGRKKRRQRRGTPQSSKDHQNPIPKQPIPQTQGVSTGPEESKKKVESKAETDRFD

SEQ ID NO: 15 - DNA, 306 bp, Human immunodeficiency virus
  CDS (1)..(306)
atggagccagtagatcctagactagagccctggaagcatccaggaagtcagcctaagactgcttgtaccaattgctattgtaaaaagtgttgctttcattgccaagtttgtttcataacaaaaggcttaggcatctcctatggcaggaagaagcggagacagcgacgaagagctcctcaagacagtcagactcatcaagtttctctatcaaagcaacccgcctcccagccccgaggggacccgacaggcccgaaggaatcgaagaagaaggtggagagagagacagagacagatccggtcgattag

SEQ ID NO: 16 - protein, 101 aa, Human immunodeficiency virus
MEPVDPRLEPWKHPGSQPKTACTNCYCKKCCFHCQVCFITKGLGISYGRKKRRQRRRAPQDSQTHQVSLSKQPASQPRGDPTGPKESKKKVERETETDPVD

SEQ ID NO: 17 - DNA, 306 bp, Human immunodeficiency virus
  CDS (1)..(306)
atggagccagtagatcctaacctagagccctggaaccatccaggaagtcagcctaaaactgcttgtaataagtgttattgtaaacactgtagctatcattgtctagtttgctttcagacaaaaggcttaggcatttcctatggcaggaagaagcggagacagcgacgaagcgctcctccaagcagtgaggatcatcaaaatcttatatcaaagcaacccttaccccaaacccaaggggacccgacaggctcggaagaatcgaagaagaaggtggagagcaagacagagacagatccattcgattag

SEQ ID NO: 18 - protein, 101 aa, Human immunodeficiency virus
MEPVDPNLEPWNHPGSQPKTACNKCYCKHCSYHCLVCFQTKGLGISYGRKKRRQRRSAPPSSEDHQNLISKQPLPQTQGDPTGSEESKKKVESKTETDPFD

SEQ ID NO: 19 - DNA, 261 bp, Human immunodeficiency virus
  CDS (1)..(261)
atggatccagtagatcctaacctagagccctggaaccatccaggaagtcagcctaggactccttgtaacaagtgttattgtaaaaagtgttgctatcattgccaagtttgcttcataacgaaaggcttaggcatctcctatggcaggaagaagcggagacagcgacgaagacctcctcaaggcggtcaggctcatcaagatcctataccaaagcaaccctcctcccagccccgaggggacccgacaggcccgaaggaatag

SEQ ID NO: 20 - protein, 86 aa, Human immunodeficiency virus
MDPVDPNLEPWNHPGSQPRTPCNKCYCKKCCYHCQVCFITKGLGISYGRKKRRQRRRPPQGGQAHQDPIPKQPSSQPRGDPTGPKE

SEQ ID NO: 21 - DNA, 306 bp, Human immunodeficiency virus
  CDS (1)..(306)
atggaactagtagatcctaacttagatccctggaaccatccaggaagccagcctacaactccttgtaccaaatgctattgtaaaaggtgttgctttcattgccaatggtgctttacaacgaagggcttaggcatctcctatggcaggaagaagcggagacagcgacgaagaactcctcaaagcagtcagatacatcaagatcctgtaccaaagcaacccttatcccaagcccgagggaacccgacaggcccgaaggaatcgaagaaggaggtggagagcaaggcaaagacagatccgtgcgattag

SEQ ID NO: 22 - protein, 101 aa, Human immunodeficiency virus
MELVDPNLDPWNHPGSQPTTPCTKCYCKRCCFHCQWCFTTKGLGISYGRKKRRQRRRTPQSSQIHQDPVPKQPLSQARGNPTGPKESKKEVESKAKTDPCD

SEQ ID NO: 23 - DNA, 306 bp, Human immunodeficiency virus
  CDS (1)..(306)
atggacccggtagatcctaacctagagccctggaatcatccggggagtcagcctaaaactccctgtaacaaatgttattgtaaaatgtgttgctggcattgtcaagtttgctttctgaacaaaggcttaggcatctcctatggcaggaagaagcggaagcaccgacgaggaactcctcagagcagtaaggatcatcaaaatcctgtaccaaagcaacccttacccaccaccagagggaacccgacaggcccgaaggaatcgaagaaggaggtggagagcaagacagagacagatccattcgattag

SEQ ID NO: 24 - protein, 101 aa, Human immunodeficiency virus
MDPVDPNLEPWNHPGSQPKTPCNKCYCKMCCWHCQVCFLNKGLGISYGRKKRKHRRGTPQSSKDHQNPVPKQPLPTTRGNPTGPKESKKEVESKTETDPFD

SEQ ID NO: 25 - DNA, 261 bp, Human immunodeficiency virus
  CDS (1)..(261)
atggacccagtagatcctaaccaagagccctggaaccatccaggaagtcagcctaaaactgcttgtaacaattgttattgtaaaaagtgctgctatcattgccaattgtgctttttaaagaaaggcttaggcatttcctatggcaggaagaagcggagccagcgacgaggaactcctgcaagtttgcaagatcatcaaaatcctataccaaagcaacccttatcccgaacccgcggggacccgacaggcccgaaggaatag

SEQ ID NO: 26 - protein, 86 aa, Human immunodeficiency virus
MDPVDPNQEPWNHPGSQPKTACNNCYCKKCCYHCQLCFLKKGLGISYGRKKRSQRRGTPASLQDHQNPIPKQPLSRTRGDPTGPKE

SEQ ID NO: 27 - DNA, 306 bp, Human immunodeficiency virus
  CDS (1)..(306)
atggagctggtagatcctaacctagagccctggaatcatccgggaagtcagcctacaactgcttgtagcaagtgttactgtaaaatatgttgctggcattgccaactatgctttctgaaaaaaggcttaggcatctcctatggcaggaagaagcggaagcaccgacgaggaactcctcagagcagtaaggatcatcaaaatcctataccagagcaacccctacccatcatcagagggaacccgacagacccgaaagaatcgaagaaggaggtggcgagcaaggcagagacagatccgtgcgattag

SEQ ID NO: 28 - protein, 101 aa, Human immunodeficiency virus
MELVDPNLEPWNHPGSQPTTACSKCYCKICCWHCQLCFLKKGLGISYGRKKRKHRRGTPQSSKDHQNPIPEQPLPIIRGNPTDPKESKKEVASKAETDPCD

SEQ ID NO: 29 - DNA, 306 bp, Human immunodeficiency virus
  CDS (1)..(306)
atggagccggtagatcctagcctagagccctggaaccacccgggaagtcagcctacaactgcttgtagcaattgttactgtaaaatgtgctgctggcattgccaattgtgctttctgaacaagggcttaggcatctcctatggcaggaagaagcggagacgccgacgaggaactcctcagagccgtcaggatcatcaaaatcctgtaccaaagcaacccttacccaccaccagagggaacccgacaggcccgaaagaatcgaagaaggaggtggcgagcaagacagagacagatccgtgcgattag

SEQ ID NO: 30 - protein, 101 aa, Human immunodeficiency virus
MEPVDPSLEPWNHPGSQPTTACSNCYCKMCCWHCQLCFLNKGLGISYGRKKRRRRRGTPQSRQDHQNPVPKQPLPTTRGNPTGPKESKKEVASKTETDPCD

SEQ ID NO: 31 - DNA, 348 bp, Human immunodeficiency virus
  CDS (1)..(348)
atggatccagtagatcctgagatgcccccttggcatcaccctggaagtcagccccagaccccttgtaataagtgctattgcaaaagatgctgctatcattgctatgtttgttttgcaagcaagggtttgggaatctcctatggcaggaagaagcgacggagaccagccgctgctgcgagccatccagataatcaagatcctgtaccagagcaacccccatccatcaccaacaggaagcagaaacgccaggaggaacaggagaaggaggtggagaaggagacaggcccaggtggataccctcgccgcaaggattcttgccactgttgtacacggacctcaggacaataa

SEQ ID NO: 32 - protein, 115 aa, Human immunodeficiency virus
MDPVDPEMPPWHHPGSQPQTPCNKCYCKRCCYHCYVCFASKGLGISYGRKKRRRPAAAASHPDNQDPVPEQPPSITNRKQKRQEEQEKEVEKETGPGGYPRRKDSCHCCTRTSGQ

SEQ ID NO: 33 - protein, 20 aa, Human immunodeficiency virus
MEPVDPRLEPWKHPGSQPKT

SEQ ID NO: 34 - protein, 20 aa, Human immunodeficiency virus
ACTNCYCKKCCFHCQVCFIT

SEQ ID NO: 35 - protein, 15 aa, Human immunodeficiency virus
VCFITKALGISYGRK

SEQ ID NO: 36 - protein, 15 aa, Human immunodeficiency virus
SYGRKKRRQRRRPPQ

SEQ ID NO: 37 - protein, 15 aa, Human immunodeficiency virus
RRPPQGSQTHQVSLS

SEQ ID NO: 38 - protein, 21 aa, Human immunodeficiency virus
RRQRRRPPQGSQTHQVSLSKQ

SEQ ID NO: 39 - protein, 16 aa, Human immunodeficiency virus
HQVSLSKQPTSQSRGD

SEQ ID NO: 40 - protein, 14 aa, Human immunodeficiency virus
PTSQSRGDPTGPKE